(19) United States
(12) Patent Application Publication
(10) Pub. No.: US 2007/0037184 A1
(43) Pub. Date: Feb. 15, 2007

(54) METHODS AND KITS FOR EVALUATING DNA METHYLATION

(75) Inventors: VICTORIA L. BOYD, San Carlos, CA (US); KENNETH J. LIVAK, San Jose, CA (US); GERALD ZON, San Carlos, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 11/424,224

(22) Filed: Jun. 14, 2006

Related U.S. Application Data
(60) Provisional application No. 60/691,608, filed on Jun. 16, 2005.

(51) Int. Cl. C12Q 1/68; C12P 19/34

(52) U.S. Cl. 435/6; 435/91.2

(57) ABSTRACT

Methods and kits are disclosed for determining the degree of methylation of at least one target region. Typically a sample is exposed to a modifying agent to obtain a modified sample comprising a modified nucleotide. At least one target region in the modified sample is amplified. Some of the disclosed methods comprise at least one additional amplification reaction. In some embodiments, at least one mobility shifting analog is incorporated into an amplicon during an amplification reaction. The analogs are analyzed and the degree of methylation of at least one target region is determined.

### CROSS-REFERENCE TO RELATED APPLICATIONS

This application claims a priority benefit under 35 U.S.C. §119(e) from U.S. Patent Application No. 60/691,608, filed Jun. 16, 2005, the contents of which is incorporated herein by reference.

### FIELD

The present teachings generally relate to the fields of biochemistry, cell biology, and biotechnology. More specifically, methods and kits are provided for evaluating the degree of methylation of at least one genomic DNA (gDNA) target region.

### INTRODUCTION

Determining the degree of methylation of particular gDNA target regions of interest is useful in many research, diagnostic, medical, forensic, and industrial fields. The methylation of cytosine residues in gDNA is an important epigenetic alteration in eukaryotes. In humans and other mammals methylcytosine is found almost exclusively in cytosine-guanine (CpG) dinucleotides. gDNA methylation plays an important role in gene regulation and changes in methylation patterns are reportedly involved in many human cancers and certain human diseases. Among the earliest and most common genetic alterations observed in human malignancies is the aberrant methylation of CpG islands, particularly CpG islands located within the 5′ regulatory regions of genes, causing alterations in the expression of such genes. Subsequently, there is great interest in using DNA methylation markers as diagnostic indicators for early detection, risk assessment, therapeutic evaluation, recurrence monitoring, and the like (see, Widschwendter et al., Clin. Cancer Res. 10:565-71, 2004; Dulaimi et al., Clin. Cancer Res. 10:1887-93, 2004; Topaloglu et al., Clin. Cancer Res. 10:2284-88, 2004; Laird, Nature Reviews, 3:253-266, 2003; Fraga et al., BioTechniques 33:632-49, 2002; Adorjan et al., Nucleic Acids Res. 30(5):e21, 2002; and Colella et al., BioTechniques, 35(1):146-150, 2003). There is also great scientific interest in the role of DNA methylation in embryogenesis, cellular differentiation, transgene expression, transcriptional regulation, and maintenance methylation, among other things.

### SUMMARY

The present teachings are directed to methods and kits for determining the degree of methylation of at least one gDNA target region in a sample. In some embodiments, the degree of methylation of one or more target region(s) in a first sample is compared with the degree of methylation of the same target region(s) in a second sample, for example but not limited to, a biopsy sample and a control sample, a “treated” sample and an “untreated” sample, a “before” sample and an “after” sample, an embryonic sample and a newborn, juvenile, or adult sample, and so forth.

According to certain methods, at least one sample comprising at least one gDNA target region is exposed to a modifying agent to generate at least one modified sample comprising at least one target region comprising at least one modified nucleotide. A first amplification composition is formed comprising: at least some of the modified sample, a target-specific primer pair for each target region to be evaluated, and a first DNA polymerase. The first amplification composition is subjected to at least one cycle of amplification and a first amplification product is generated. According to some methods, the cycle of amplification comprises a multiplicity of amplification cycles. The first amplicons are analyzed to determine the presence or absence of at least one modified nucleotide or its complement in an amplicon and the degree of methylation for at least one target region is inferred. In some embodiments, analyzing comprises determining the number of modified nucleotides or its complement that are present in at least one first amplicon.

According to certain methods, at least one sample comprising at least one gDNA target region is exposed to a modifying agent to generate at least one modified sample comprising at least one target region comprising at least one modified nucleotide. A first amplification composition is formed comprising: at least some of the modified sample, a target-specific primer pair for each target region to be evaluated, a mobility shifting analog (MSA), and a first DNA polymerase. The first amplification composition is subjected to at least one cycle of amplification and a first amplification product comprising at least one incorporated MSA is generated. The incorporation of at least one MSA into an amplification product alters the mobility of that amplification product relative to an amplification product of the same sequence except that it does not comprise the MSA. According to some methods, the cycle of amplification comprises a multiplicity of amplification cycles. The first amplification products are analyzed to determine the presence or absence of at least one modified nucleotide or its complement and the degree of methylation for at least one target region is inferred. In some embodiments, analyzing comprises determining the number of modified nucleotides or its complement that are present in a first amplification product.

According to certain methods, at least one sample comprising at least one gDNA target region is exposed to a modifying agent to generate at least one modified sample comprising at least one target region comprising at least one modified nucleotide. A first amplification composition is formed comprising: at least some of the modified sample, a target-specific primer pair for each target region to be evaluated, and a first DNA polymerase. The first amplification composition is subjected to at least one cycle of amplification and a first amplification product is generated. According to some methods, the first cycle of amplification comprises a multiplicity of cycles of amplification. A second amplification composition is formed comprising at least some of the first amplification product, an amplification product primer, a MSA, and a second DNA polymerase. In some embodiments, the amplification product primer comprises an amplification product primer pair comprising a forward amplification product primer and a reverse amplification product primer. The second amplification composition is subjected to at least one cycle of amplification and a second amplification product comprising at least one incorporated MSA is generated. According to some methods, the second cycle of amplification comprises a multiplicity of amplification cycles. The second amplification products comprising the at least one incorporated MSA are analyzed to determine the presence or absence of a modified nucleotide or its complement and the degree of methylation for at least one target region is inferred. In some embodiments, analyzing comprises determining the number of modified nucleotides or its complement that are present in a second amplification product.

Kits for performing certain disclosed methods are also provided. In certain embodiments, kits comprise a MSA, a first DNA polymerase, and at least one target-specific primer pair. In some embodiments, kits comprise a multiplicity of different target-specific primer pairs. In certain embodiments, kits comprise at least one of: a control sequence, a modifying agent, an amplification product primer, an amplification product primer pair, a second DNA polymerase, a reporter probe, an intercalating agent, and a reporter group.

These and other features of the present teachings are set forth herein.

### DRAWINGS

The skilled artisan will understand that the drawings, described below, are for illustration purposes only. These figures are not intended to limit the scope of the present teachings in any way.

FIG. 1: provides a schematic overview of various embodiments of certain disclosed methods.

FIG. 2: depicts illustrative results obtained using a disclosed method, as described in Example 2. 1: methylated amplicon peak; 2: unmethylated amplicon peak.

FIGS. 3A & 3B: depict illustrative results obtained using a disclosed method, as described in Example 3. 1: methylated amplicon peak; 2: unmethylated amplicon peak; 10%, 25%, 50%, 75% and 100% indicate the percentage of unmethylated target region in the sample.

FIG. 4: depicts an illustrative result obtained using a disclosed embodiment comprising an exemplary MSA, alpha-thio-dCTP, as described in Example 4. 1: methylated amplicon peak; 2: unmethylated amplicon peak.

FIG. 5: depicts an illustrative result obtained using a disclosed embodiment comprising a MSA, biotin-aha-dCTP, as described in Example 4. 1: unmethylated amplicon peaks; 2: methylated amplicon peaks; lower panel: peaks “a”-“o” designate amplicon peaks derived from the exemplary methylated target region.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. For example, “a forward primer” means that more than one forward primer can be present; for example, one or more copies of a particular forward primer species, as well as one or more different forward primer species. Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including patents, patent applications, articles, books, treatises, and internet web pages are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

U.S. Provisional Patent Application No. 60/654,162, entitled “Compositions, Methods, and Kits for Analyzing DNA Methylation”, filed Feb. 18, 2005, now U.S. patent application Ser. No. 11/352,143, is hereby expressly incorporated by reference in its entirety for any purpose.

Some Definitions

The term “affinity tag” as used herein refers to a component of a multi-component complex, wherein the components of the multi-component complex specifically interact with or bind to each other. Some non-limiting examples of multiple-component affinity tag complexes include, ligands and their receptors, for example but not limited to, avidin-biotin, streptavidin-biotin, and derivatives of biotin, streptavidin, or avidin, including, 2-iminobiotin, desthiobiotin, NeutrAvidin (Molecular Probes, Eugene, Oreg.), CaptAvidin (Molecular Probes), and the like; binding proteins/peptides and their binding partners; epitope tags, for example but not limited to c-MYC, HA, VSV-G, and FLAG Tag™, and their corresponding anti-epitope antibodies; haptens, for example but not limited to dinitrophenol (“DNP”) and digoxigenin (“DIG”), and their corresponding antibodies; aptamers and their binding partners; fluorescent reporter groups and corresponding anti-fluorescent reporter group antibodies; and the like. In some embodiments, an affinity tag comprises a reporter group. In certain embodiments, an affinity tag or a hybridization tag complement may be coupled to a solid support. In certain embodiments, affinity tags and/or solid supports are part of a separating means, part of a detecting means, or both.

The terms “annealing” and “hybridizing”, including variations of the root words hybridize and anneal, are used interchangeably and mean the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions may also contribute to duplex stability.

The term “at least some of”, for example, when used in reference to a sample or a modified sample, means that all of the sample or modified sample can be used or that some, but not all, of the sample or modified sample can be used, for example, an aliquot. The term “at least part of”, for example, when used in reference to analyzing an amplification product, means that the entire amplification product can be analyzed, one or both of the individual strands of a double-stranded amplification product can be analyzed, or a fragment, portion, or subsequence of an amplification product can be analyzed.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “corresponding” as used herein refers to at least one specific relationship between the elements to which the term relates. For example, a reverse primer of a particular primer pair corresponds to the forward primer of the same primer pair, and vice versa. At least one amplification product primer is designed to anneal with the primer-binding portion of at least one corresponding amplicon. The target-specific portions of the reverse target-specific primers are designed to selectively hybridize with a complementary or substantially complementary region of the corresponding downstream target region flanking sequence. A particular affinity tag binds to the corresponding affinity tag, for example but not limited to, biotin binding to streptavidin. A particular hybridization tag anneals with its corresponding hybridization tag complement; and so forth.

As used herein, the term “degree of methylation” when used in reference to a gDNA target region, refers to the amount of that target region within a sample that is methylated relative to the amount of the same target region that is not methylated, or to the relative number of methylated nucleotides in a target region, or both. In certain embodiments, a sample contains a target region that is fully methylated, a target region that is unmethylated, a target region that has some copies that are fully methylated and some copies that are unmethylated. In some embodiments, a sample comprises copies of a target region that have some but not all of its target nucleotides methylated (intermediate methylation), including some copies with one amount of intermediate methylation and some other copies with at least one different level of intermediate methylation. In some embodiments, determining the degree of methylation for a particular target region comprises obtaining the ratio of methylated target region to unmethylated target region, for example but not limited to, the ratio between the peak height of an amplicon derived from a methylated target region relative to the peak height of an amplicon derived from the same, but unmethylated target region. In certain embodiments, determining the degree of methylation for a particular target region comprises identifying the number or methylated nucleotides in the target region, for example but not limited to evaluating the incremental mobility shift of an amplicon comprising at least one MSA and calculating the number of incorporated MSAs based on the size of the incremental mobility shift to determine the number of methylated nucleotides in the target region from which the amplicon was derived.

The terms “denaturing” or “denaturation” as used herein refer to any process in which a double-stranded polynucleotide, including a double-stranded amplification product or a double-stranded gDNA fragment is converted to two single-stranded polynucleotides. Denaturing a double-stranded polynucleotide includes without limitation, a variety of thermal and chemical techniques for denaturing a duplex, thereby releasing its two single-stranded components. Those in the art will appreciate that the denaturing technique employed is generally not limiting unless it inhibits or appreciably interferes with a subsequent amplifying and/or determining step.

The term “DNA polymerase” is used in a broad sense herein and refers to any polypeptide that is able to catalyze the addition of deoxyribonucleotides or analogs of deoxyribonucleotides to a nucleic acid polymer in a template dependent manner. For example but not limited to, the sequential addition of deoxyribonucleotides to the 3′-end of a primer that is annealed to a nucleic acid template during a primer extension reaction. Typically DNA polymerases include DNA-dependent DNA polymerases and RNA-dependent DNA polymerases, including reverse transcriptases. Certain reverse transcriptases possess DNA-dependent DNA polymerase activity under certain reaction conditions, including AMV reverse transcriptase and MMLV reverse transcriptase. Such reverse transcriptases with DNA-dependent DNA polymerase activity may be suitable for use with the disclosed methods and are expressly within the contemplation of the current teachings. Descriptions of DNA polymerases can be found in, among other places, Lehninger Principles of Biochemistry, 3d ed., Nelson and Cox, Worth Publishing, New York, N.Y., 2000, particularly Chapters 26 and 29; Twyman, Advanced Molecular Biology: A Concise Reference, Bios Scientific Publishers, New York, N.Y., 1999; Ausubel et al., Current Protocols in Molecular Biology, John Wiley & Sons, Inc., including supplements through May 2005 (hereinafter “Ausubel et al.”); Lin and Jaysena, J. Mol. Biol. 271:100-11, 1997; Pavlov et al., Trends in Biotechnol. 22:253-60, 2004; and Enzymatic Resource Guide: Polymerases, 1998, Promega, Madison, Wis. Expressly within the intended scope of the term DNA polymerase are enzymatically active mutants or variants thereof, including enzymes modified to confer different temperature-sensitive properties (see, e.g. U.S. Pat. Nos. 5,773,258; 5,677,152; and 6,183,998; and DNA Amplification: Current Techniques and Applications, Demidov and Broude, eds., Horizon Bioscience, 2004, particularly in Chapter 1.1).

The term “intercalating agent” is used in a broad sense and includes any compound or material that: (1) can be (a) inserted between the nucleotide bases of a polynucleotide or (b) between the strands of a polynucleotide (sometimes referred to as a groove binder) and (2) is detectable, directly or indirectly. Non-limiting examples of intercalating agents include certain antibiotic and anti-tumor substances such as actinomycin D, hycanthone, berenil, netropsin, distamycin, bleomycin, and daunomycin which may or may not further comprise a reporter group, psoralen, intercalating dyes such as acridine orange, diaminoacridine, SYBR® Green, proflavine, ethidium bromide, thiazole orange (TO) family dyes including TOTO dyes, POPO family dyes, oxazole yellow (YO) family dyes including YOYO dyes, DAPI, Hoechst 33258, propidium iodide (PI), and chloroquine, and including derivatives of such compounds. Descriptions of intercalating agents including groove binders can be found in, among other places, Nucleic Acids in Chemistry and Biology, 2^{nd} Ed., Blackburn and Gait, eds., 1996, Oxford University Press, Oxford U.K. (hereinafter “Blackburn and Gait”), particularly in sections 8.3-8.5; and Haugland, The Handbook, A Guide to Fluorescent Probes and Labeling Technologies, 10th Ed., 2005, Invitrogen Corp., Carlsbad, Calif. (hereinafter “Haugland”).

The term “methylated amplicon” refers to an amplification product that is derived from a target region that comprises at least one methylated target nucleotide, for example but not limited to a 5 mC. A methylated amplicon can be either double-stranded or single-stranded and can be a first amplification product, a second amplification product, or both. The term “unmethylated amplicon” refers to an amplification product that is derived from a target region that does not comprise a methylated target nucleotide. An unmethylated amplicon can be either double-stranded or single-stranded and can be a first amplification product, a second amplification product, or both.

In certain embodiments, a gDNA sample comprising at least one target region is treated with a modifying agent to obtain a modified sample comprising at least one modified target nucleotide. The term “modifying agent” refers to any reagent that can modify a nucleic acid, for example but not limited to at least one target nucleotide in at least one gDNA target region. Some modifying agents convert an unmethylated target nucleotide to a modified nucleotide, but do not convert a methylated target nucleotide to a modified nucleotide (at least not to a significant degree).

In certain embodiments, bisulfite is employed as a modifying agent. Incubating nucleic acid sequences such as gDNA with bisulfite results in deamination of a substantial portion of unmethylated cytosines, which converts such cytosines to uracil. Methylated cytosines are deaminated to a measurably lesser extent. In certain embodiments, the sample is then amplified, resulting in the uracil bases being replaced with thymine. Thus, in certain embodiments, a substantial portion of unmethylated target cytosines ultimately become thymines, while a substantial portion of methylated cytosines remain cytosines. In certain embodiments, the presence of a modified nucleotide (for example but not limited to, uracil or thymine) in the target region may be determined using the methods and kits of the present teachings. Descriptions of bisulfite treatment can be found in, among other places, U.S. Pat. Nos. 6,265,171 and 6,331,393; Boyd and Zon, Anal. Biochem. 326: 278-280, 2004; U.S. Provisional Patent Application Ser. Nos. 60/499,113; 60/520,942; 60/499,106; 60/523,054; 60/498,996; 60/520,941; 60/499,082; and 60/523,056.

The term “nucleotide terminator” or “terminator” refers to an enzymatically-incorporable nucleotide, which does not support incorporation of subsequent nucleotides in an amplifying reaction and is therefore not an extendable nucleotide. In some embodiments, a first amplification composition and/or a second amplification composition comprises a nucleotide terminator. In some embodiments, a nucleotide terminator comprises a reporter group.

The term “reporter group” is used in a broad sense herein and refers to any identifiable tag, label, or moiety. The skilled artisan will appreciate that many different species of reporter groups can be used in the present teachings, either individually or in combination with one or more different reporter group.

The term “selectively hybridize” and variations thereof means that, under suitable conditions, a given sequence anneals with a second sequence comprising a complementary or a substantially complementary string of nucleotides, but does not anneal to undesired sequences. In this application, a statement that one sequence selectively hybridizes or anneals with another sequence encompasses situations where the entirety of both of the sequences hybridize to one another, and situations where only a portion of one or both of the sequences hybridizes to the entire other sequence or to a portion of the other sequence. For the purposes of this definition, the term “sequence” includes nucleic acid sequences, polynucleotides, oligonucleotides, primers, target-specific portions, amplification product-specific portions, primer-binding sites, hybridization tags, and hybridization tag complements.

In this application, a statement that one sequence is the same as, substantially the same as, complementary to, or substantially complementary to another sequence encompasses situations where both of the sequences are completely the same as, substantially the same as, or complementary or substantially complementary to one another, and situations where only a portion of one of the sequences is the same as, substantially the same as, complementary to, or substantially complementary to a portion or the entire other sequence. For the purposes of this definition, the term “sequence” includes nucleic acid sequences, polynucleotides, oligonucleotides, primers, target-specific portions, amplification product-specific portions, primer-binding sites, hybridization tags, and hybridization tag complements.

Certain Exemplary Components

The term “sample” is used in a broad sense herein and is intended to include a wide range of biological materials as well as compositions derived or extracted from such biological materials comprising or suspected of comprising gDNA. Exemplary samples include whole blood; nucleated red blood cells; white blood cells; buffy coat; hair; nails and cuticle material; swabs, including buccal swabs, throat swabs, vaginal swabs, urethral swabs, cervical swabs, rectal swabs, lesion swabs, abcess swabs, nasopharyngeal swabs, and the like; urine; sputum; saliva; semen; lymphatic fluid; amniotic fluid; cerebrospinal fluid; peritoneal effusions; pleural effusions; fluid from cysts; synovial fluid; vitreous humor; aqueous humor; bursa fluid; eye washes; eye aspirates; plasma; pulmonary ravages; lung aspirates; and tissues, including, liver, spleen, kidney, lung, intestine, brain, heart, muscle, pancreas, biopsy material, and the like. The skilled artisan will appreciate that lysates, extracts, or material obtained from any of the above exemplary biological samples are also within the scope of the current teachings. Tissue culture cells, including explanted material, primary cells, secondary cell lines, and the like, as well as lysates, extracts, or materials obtained from any cells, are also within the meaning of the term sample as used herein. Materials comprising or suspected of comprising at least one gDNA target region that are obtained from forensic, agricultural, and/or environmental settings are also within the intended meaning of the term sample. In certain embodiments, a sample comprises a synthetic nucleic acid sequence. In some embodiments, a sample is totally synthetic, for example but not limited to a control sample comprising a buffer solution containing at least one synthetic nucleic acid sequence.

The first amplification compositions of the current teachings comprise gDNA that includes at least one target region located between a corresponding first flanking sequence and a second flanking sequence. The “first target flanking sequence” is typically located upstream from, i.e., on the 5′ side of, the target region and the corresponding “second target flanking sequence” is typically located downstream from, i.e., on the 3′ side of, the target region. For illustration purposes, the orientation of an illustrative target region relative to its two target flanking sequences is: 5′-first target flanking sequence-target region-second target flanking sequence-3′. It is to be understood that the target flanking sequences can, but need not, be contiguous with the target region. Thus, additional nucleotides may be present between a target flanking sequence and the target region. The target-binding portion of the forward target-specific primer comprises a sequence that is designed to selectively hybridize with the complement of the first target flanking sequence or a sub-sequence within the first target flanking sequence. The target-binding portion of the reverse target-specific primer comprises a sequence that is designed to selectively hybridize with the second target flanking sequence or a sub-sequence within the second target flanking sequence.

The term “target region” refers to the gDNA segment that is being amplified and analyzed to determine the presence or absence of methylated nucleotides and infer the degree of target region methylation. A target region may be located in the promoter or regulatory elements of a gene of interest that is known or suspected of being methylated under certain physiological conditions. The target region is generally located between two flanking sequences, a first target flanking region and a second target flanking region, located on either side of, but not necessarily immediately adjacent to, the target region. In some embodiments, a gDNA segment comprises a plurality of different target regions. In some embodiments, a target region is contiguous with or adjacent to one or more different target regions. In some embodiments, a given target region can overlap a first target region on its 5′-end, a second target region on its 3′-end, or both.

A target region can be either synthetic or naturally occurring. Certain target regions, including flanking sequences where appropriate, can be synthesized using oligonucleotide synthesis methods that are well-known in the art. Detailed descriptions of such techniques can be found in, among other places, Current Protocols in Nucleic Acid Chemistry, Beaucage et al., eds., John Wiley & Sons, New York, N.Y., including updates through May 2005 (hereinafter “Beaucage et al.”); and Blackburn and Gait. Automated DNA synthesizers useful for synthesizing target regions and primers are commercially available from numerous sources, including for example, the Applied Biosystems DNA Synthesizer Models 381A, 391, 392, and 394 (Applied Biosystems, Foster City, Calif.). Target regions, including flanking regions where appropriate, can also be generated biosynthetically, using in vivo methodologies and/or in vitro methodologies that are well known in the art. Descriptions of such technologies can be found in, among other places, Sambrook et al., Molecular Cloning, A Laboratory Manual, Cold Spring Harbor Press (1989) (hereinafter “Sambrook et al.”); and Ausubel et al. Genomic DNA can also be obtained from biological materials using any sample preparation technique known in the art. Purified or partially purified gDNA is commercially available from numerous sources, including Coriell Cell Repositories, Coriell Institute for Medical Research, Camden, N.J.; Serologicals Corp., Norcross, Ga.; and the American Type Culture Collection (ATCC), Manassas, Va.

As used herein, the terms “polynucleotide”, “oligonucleotide”, and “nucleic acid” are used interchangeably and refer to single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺, and the like. A polynucleotide may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytosine or possibly 5-methyldeoxycytosine (5 mC), “G” denotes deoxyguanosine, “T” denotes thymidine, and “U” denotes deoxyuridine, unless otherwise noted.

The tern “nucleotide base”, as used herein, refers to a substituted or unsubstituted aromatic ring or rings. In certain embodiments, the aromatic ring or rings contain a nitrogen atom. In certain embodiments, the nucleotide base is capable of forming Watson-Crick or Hoogsteen-type hydrogen bonds with a complementary nucleotide base. Exemplary nucleotide bases and analogs thereof include, naturally-occurring nucleotide bases adenine, guanine, cytosine, 5 mC, uracil, and thymine, and analogs of the naturally occurring nucleotide bases, including, 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7 mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT Published Application WO 01/38584), ethenoadenine, indoles such as nitroindole and 4-methylindole, and pyrroles such as nitropyrrole. Non-limiting examples of nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different, —R, —OR, —NR₂ azide, cyanide or halogen groups, where each R is independently H, C₁-C₆ alkyl, C₂-C₇ acyl, or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′, 3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT Published Application Nos. WO 98/22489, WO 98/39352, and WO 99/14226; and Braasch and Corey, Chem. Biol. 8:1-7, 2001). “LNA” or “locked nucleic acid” is a DNA analogue that is conformationally locked such that the ribose ring is constrained by a methylene linkage between, for example but not limited to, the 2′-oxygen and the 3′- or 4′-carbon or a 3′-4′ LNA with a 2′-5′ backbone (see, e.g., U.S. Pat. Nos. 6,268,490 and 6,670,461). The conformation restriction imposed by the linkage often increases binding affinity for complementary sequences and increases the thermal stability of such duplexes. Exemplary LNA sugar analogs within a polynucleotide include the structures: 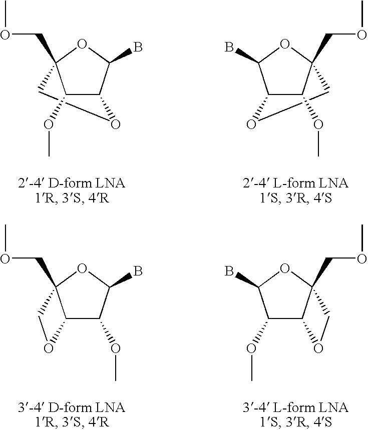
where B is any nucleotide base.

The 2′- or 3′-position of ribose can be modified to include hydrogen, hydroxy, methoxy, ethoxy, allyloxy, isopropoxy, butoxy, isobutoxy, methoxyethyl, alkoxy, phenoxy, azido, cyano, amido, imido, amino, alkylamino, fluoro, chloro and bromo. Nucleotides include the natural D optical isomer, as well as the L optical isomer forms (see, e.g., Garbesi et al., Nucl. Acids Res. 21:4159-65 (1993); Fujimori et al., J. Amer. Chem. Soc. 112:7436-38, 1990; Urata et al., Nucl. Acids Symposium Ser. No. 29:69-70 (1993)). When the nucleotide base is a purine, e.g., A or G, the ribose sugar is attached to the N⁹-position of the nucleotide base. When the nucleotide base is a pyrimidine, e.g. C, T, or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula: 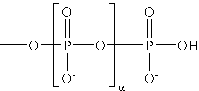
where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and is sometimes denoted as “rNTP”, or “dNTP” and “ddNTP” to particularly point out the structural features of the ribose sugar, or generically as “NTP”. The triphosphate ester group may include sulfur substitutions for the various oxygens, e.g., α-thio-nucleotide 5′-triphosphates. Reviews of nucleotide chemistry can be found in, among other places, Shabarova, Z. and Bogdanov, A. Advanced Organic Chemistry of Nucleic Acids, VCH, New York, 1994; and Blackburn and Gait.

The term “nucleotide analog”, as used herein, refers to embodiments in which the pentose sugar or the nucleotide base or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boranophosphates, etc., and may include associated counterions.

The term “mobility shifting analog” or “MSA” refers to a nucleotide analog of dATP, dCTP, dGTP, dUTP, or dTTP, that when incorporated into an amplicon detectably changes the migration rate or the amplicon in an analyzing technique, such as a mobility dependent analysis technique, relative to an amplicon comprising the same sequence but with the natural nucleotides not the MSA(s). In other words, the amplicon comprising the incorporated MSA migrates at a different position in at least one analysis technique than would be expected from its length. In some embodiments, an amplicon comprising a MSA migrates faster than its counterpart lacking the MSA. In other embodiments, an amplicon comprising an MSA migrates more slowly that its counterpart lacking the MSA. Non-limiting examples of nucleotide analogs that may be suitable for inducing a mobility shift include boranotriphosphates (including α-P-boranotriphosphates), thiotriphosphates (including deoxy-5′-(α-thio)triphosphate, e.g., dCTPαS), nucleotide analogs comprising long linker arms, for example but not limited to, (CH₂)n and/or (OCH₂CH₂)n, including biotin-11-dCTP, biotin-11-dCTP, biotin-11-dUTP, digoxigenin-11-dUTP, biotin-aminohexylacrylamido-dCTP (biotin-aha-dCTP), biotin-aha-dUTP, biotin-14-dCTP, biotin-36-dUTP, biotin-36-dCTP, biotin-36-dcATP, and heterocycles, for example but not limited to biotin, N-substituted biotin, and homobiotin cognates, heterocyclic derivatives of hydrocarbon, and polyethylene glycol cognates. Those in the art will appreciate that the suitability of a particular nucleotide analog for use as a MSA depends at in part on the target region, the DNA polymerase used for the amplification reaction, the mobility shift imparted by each analog, the separation and/or detection means, the software, or combinations thereof. Those in the art will understand that the suitability of one or more MSAs can be empirically evaluated, for example using target regions of known methylation state as the starting materials and performing one or more of the disclosed methods under the desired or various reaction conditions, without undue experimentation.

The term “primer” refers to a polynucleotide that selectively hybridizes to a gDNA target flanking sequence or to a corresponding primer-binding site of an amplification product; and allows the synthesis of a sequence complementary to the corresponding polynucleotide template from its 3′ end.

A “target-specific primer pair” of the current teachings comprises a forward target-specific primer and a reverse target-specific primer. The forward target-specific primer comprises a first target-specific portion that comprises a sequence that is the same as or substantially the same as the nucleotide sequence of the first or upstream target flanking sequence, and that is designed to selectively hybridize with the complement of the upstream target flanking sequence that is present in, among other places, the reverse strand amplification product. In some embodiments, the forward target-specific primer further comprises a first tail portion, located upstream from the first target-specific portion, that comprises a first primer-binding site. The reverse target-specific primer of the primer pair comprises a second target region-specific portion that comprises a sequence that is complementary to or substantially complementary to, and that is designed to selectively hybridize with, the second or downstream target region flanking sequence. In some embodiments, the reverse target-specific primer further comprises a second tail portion, located upstream from the second target-specific portion, that comprises a second primer-binding site. In some embodiments, the tail portion of a reverse target-specific primer further comprises a sequence that is designed to enhance the non-templated addition of nucleotides, typically A, to the end of a primer extension product by certain DNA polymerases, sometimes referred to as the Clark reaction (see, e.g., Clark, Nucl. Acids Res. 16(20):9677-84, 1988). Some non-limiting examples of such sequences include GTTTCTT, GTTT, and GTT, sometimes referred to as PIGtail sequences (see, e.g., Brownstein et al., BioTechniques 20(6): 1004-10, 1996), or a single G at the 5′-end of a tailed primer. In certain embodiments, at least one forward target-specific primer, at least one reverse target-specific primer, or at least one forward target-specific primer and at least one reverse target-specific primer further comprises at least one of: a reporter probe-binding site, an additional primer-binding site, or a reporter group, for example but not limited to a fluorescent reporter group. In certain embodiments, a forward primer and the corresponding reverse primer of a target-specific primer pair have different melting temperatures (Tm) to permit temperature-based asymmetric PCR.

In some embodiments, a target-specific primer pair comprises (1) a forward target-specific primer comprising a first target-binding portion that is the same as or substantially the same as a first target flanking sequence, located upstream (5′) of the gDNA target region and (2) a corresponding reverse target-specific primer comprising a second target-binding portion that is complementary to or substantially complementary to a corresponding second target flanking sequence, located downstream (3′) of the same gDNA target region. In some embodiments, a target-specific primer pair, includes (1) a forward target-specific primer comprising (a) a first target-binding portion that is the same as or substantially the same as a first target flanking sequence, located upstream (5′) of the gDNA target region and (b) a first tail portion located upstream from the first target-binding portion, wherein the tail sequence comprises a first primer-binding site; and (2) a corresponding reverse target-specific primer comprising (a) a second target-binding portion that is complementary to or substantially complementary to a corresponding second target flanking sequence, located downstream (3′) of the same gDNA target region and (b) a second tail sequence located upstream from the second target-binding sequence, wherein the second tail sequence comprises a second primer-binding site.

Those in the art will appreciate that treatment of gDNA with certain modifying agents, for example but not limited to sodium bisulfite, cause unmethylated C to be deaminated to U. A gDNA flanking region comprising an unmethylated C would, after sodium bisulfite treatment, result in a modified nucleotide in the flanking region of modified sample, which could prevent or decrease the ability of the corresponding target-specific primer to selectively hybridize. The target-specific primers of the current teachings are typically designed to selectively hybridize with target flanking sequences that are outside CpG islands to allow a target region amplicon to be generated regardless of the methylation state of the target region.

The term “amplification product primer pair” refers to a forward amplification product primer and a corresponding reverse amplification product primer. In some embodiments, an amplification primer pair comprises a universal primer or a universal primer pair and the same primer pair is used to amplify at least two different species of amplification product. In some embodiments, an amplification product primer pair comprises a forward primer and a reverse primer that are designed to amplify one amplification product species. For example but without limitation, a first amplification product primer pair comprising a forward first amplification product primer comprising a sequence that is designed to selectively hybridize with the complement of an upstream primer-binding site of a particular single-stranded first amplification product species and a reverse first amplification product primer that is designed to selectively hybridize with the corresponding downstream primer-binding site of the same single-stranded first amplification product species. In some embodiments, an amplification product primer pair is designed to selectively hybridize with corresponding regions of an amplification product or its complement that are internal to the binding sites of the target-specific primer pair, including a nested primer pair, or to binding sites that partially overlap the binding sites of the target-specific primer pair. In certain embodiments, at least one forward amplification product primer, at least one reverse amplification product primer, or at least one forward amplification product primer and at least one reverse amplification product primer further comprises at least one of: a reporter probe-binding site, an additional primer-binding site, and a reporter group, for example but not limited to a fluorescent reporter group. In certain embodiments, a forward primer and the corresponding reverse primer of an amplification product primer pair have different melting temperatures to permit temperature-based asymmetric PCR.

In certain embodiments, one or more of a primer's components may overlap or partially overlap one or more other primer components. For example but not limited to, a target-specific portion may overlap or partially overlap a primer-binding site, a reporter probe-binding site, a hybridization tag, an affinity tag, a reporter group.

The skilled artisan will appreciate that the complement of the disclosed gDNA target regions, primers, target-specific portions, primer-binding sites, or combinations thereof, may be employed in certain embodiments of the present teachings. For example, without limitation, a particular gDNA may comprise both the gDNA target region and its complement. Thus, in certain embodiments, when a gDNA sample is denatured, both the target region and its complement are present in the sample as single-stranded sequences and either or both of the single-stranded sequences can be amplified and analyzed. Those in the art will appreciate, however, that in certain circumstances, a double-stranded gDNA segment comprising a target region may be hemimethylated. For example, but not as a limitation, one strand of the double-stranded gDNA segment may comprise a methylated target nucleotide while the corresponding target nucleotide in the complementary gDNA strand is unmethylated. In certain embodiments, it is desirable to determine the degree of methylation of both the target region and its complement to obtain an accurate understanding of the methylation state of the gDNA segment in question.

As used herein, the terms “forward” and “reverse” are used to indicate relative orientation of the corresponding primers of a primer pair on a polynucleotide sequence. For illustration purposes but not as a limitation, consider a single-stranded polynucleotide drawn in a horizontal, left to right orientation with its 5′-end on the left. The “reverse” primer is designed to anneal with the downstream primer-binding site at or near the “3′-end” of this illustrative polynucleotide, in a 5′ to 3′ orientation, right to left. The corresponding “forward primer is designed to anneal with the complement of the upstream primer-binding site at or near the “5′-end” of the polynucleotide, in a 5′ to 3′ “forward” orientation, left to right. Thus, the reverse primer comprises a sequence that is complementary to the reverse or downstream primer-binding site of the polynucleotide and the forward primer comprises a sequence that is the same as the forward or upstream primer-binding site. It is to be understood that the terms “3′-end” and “5′-end”, as used in this paragraph, are illustrative only and do not necessarily refer literally to the respective ends of the polynucleotide. Rather, the only limitation is that the reverse primer of this exemplary primer pair anneals with a reverse primer-binding site that is downstream or to the right of the forward primer-binding site that comprises the same sequence or substantially the same sequence as the corresponding forward primer. As will be recognized by those of skill in the art, these terms are not intended to be limiting, but rather to provide illustrative orientation in a given embodiment.

As used herein, the term “primer-binding site” refers to a region of a polynucleotide sequence such as a tailed primer or an amplification product that can serve directly, or by virtue of its complement, as the template upon which a primer can anneal for any of a variety of primer extension reactions known in the art, for example but not limited to, PCR. When a tailed primer comprises a primer-binding site, it is typically located upstream from a sequence-specific binding portion of the primer, for example but not limited to, the first target-binding portion of a forward target-specific primer or the second primer-binding portion of a reverse amplification product primer.

Those in the art appreciate that as an amplification product is amplified by certain amplification techniques, the complement of the primer-binding site is synthesized in the complementary strand. Thus, it is to be understood that the complement of a primer-binding site is expressly included within the intended meaning of the term primer-binding site, unless stated otherwise.

In some embodiments, a primer-binding site comprises a universal priming sequence or its complement, allowing at least some amplification products to be generated using a universal primer or a pair of universal primers. In some embodiments, a sequencing primer comprises a universal priming sequence. A “universal primer” is capable of selectively hybridizing to the corresponding primer-binding site of more than one species of amplification product. A “universal primer pair” comprises a forward universal primer and a reverse universal primer that are designed to hybridize with a plurality of species of amplification products. In certain embodiments, a universal primer or a universal primer pair selectively hybridizes with all or most of the amplification products in a reaction. Universal primers/priming sequences (sometimes referred to as common or generic primers), including M13 universal primers and T7 universal primers, and their use are well known in the art (see, e.g., McPherson and Moller, PCR The Basics, Bios Scientific Publishers, Oxford, U.K., 2000 (hereinafter “McPherson”), particularly section 4.2 of Chapter 5). In some embodiments, a universal primer or a pair of universal primers can be employed as sequencing primers for a sequencing reaction; and either or both strands of a double-stranded amplification product can be sequenced. Universal primers are commercially available from numerous vendors including Applied Biosystems, USB Corporation, Invitrogen, and Promega. Those in the art will understand that “custom” universal primers can also be designed and synthesized using methods known in the art.

In some embodiments, a multiplicity of different primer pairs are employed in an amplifying step, for example but not limited to a multiplex amplification reaction, wherein the different primer pairs are designed to amplify a multiplicity of different nucleotide sequences, including a multiplicity of different gDNA target regions or a multiplicity of different amplification products.

Conditions under which primers selectively hybridize to complementary or substantially complementary sequences are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349, 1968. In general, whether such annealing takes place is influenced by, among other things, the length of the complementary portion of the primers and their corresponding target region flanking sequences or the corresponding primer-binding sites in amplification products, the pH, the temperature, the presence of mono- and divalent cations, the proportion of G and C nucleotides in the hybridizing region, the viscosity of the medium, and the presence of denaturants. Such variables influence the time required for hybridization. The presence of certain nucleotide analogs or minor groove binders in the sequence-specific portion of a primer and/or a corresponding amplification product can also influence hybridization conditions. Thus, the preferred annealing conditions will depend upon the particular application. Such conditions, however, can be routinely determined by persons of ordinary skill in the art, without undue experimentation. Typically, annealing conditions are selected to allow the disclosed primers to selectively hybridize with a complementary or substantially complementary sequence in a corresponding target region flanking sequence or a corresponding amplification product, but not hybridize to any significant degree to other undesired sequences in the reaction.

The sequence-specific portions of the disclosed primers are of sufficient length to permit specific annealing with complementary or substantially complementary sequences in target flanking sequences and/or amplicons, as appropriate. The criteria for designing sequence-specific primers are well known to persons of ordinary skill in the art. Descriptions of primer design can be found in, among other places, Diffenbach and Dveksler, PCR Primer, A Laboratory Manual, Cold Spring Harbor Press (1995); Rapley, The Nucleic Acid Protocols Handbook (2000), Humana Press, Totowa, N.J. (hereinafter “Rapley”); and Kwok et al., Nucl. Acid Res. 18:999-1005 (1990). Primer design software programs are also commercially available, for example, Primer Premier 5, PREMIER Biosoft, Palo Alto, Calif.; Primer Designer 4, Sci-Ed Software, Durham, N.C.; Primer Detective, ClonTech, Palo Alto, Calif.; Lasergene, DNASTAR, Inc., Madison, Wis.; and iOligo, Caesar Software, Portsmouth, N.H.

The skilled artisan will appreciate that while the primers and primer pairs of the present teachings may be described in the singular form, a plurality of primers may be encompassed by the singular term. Thus, for example, in certain embodiments, a target-specific primer pair typically comprises a plurality of forward target-specific primers and a plurality of corresponding reverse target-specific primers.

Those in the art understand that primers and primer pairs that are suitable for use with the disclosed methods and kits can be identified empirically using the current teachings and routine methods known in the art, without undue experimentation. For example, suitable target regions, including appropriate flanking sequences can be obtained by searching relevant scientific literature, including appropriate databases, that list or identify known or suspected hyper- or hypo-methylation sites; or by experimental analysis. When target regions and appropriate flanking regions are identified, test primers can be synthesized using well known oligonucleotide synthesis techniques (see, e.g., Beaucage et al.; Blackburn and Gait; Glen Research 2002 Catalog, Sterling, Va.; and Synthetic Medicinal Chemistry 2003/2004, Berry and Associates, Dexter, Mich.). Test primers can be employed according to the current teachings and their suitability for generating amplicons can be evaluated. Standard curves can be generated, if desired, using pre-determined mixtures or serial dilutions of synthetic templates or gDNA sequences of known methylation state, using a method of the current teachings and under standard conditions.

In some embodiments, a forward primer, a reverse primer, a MSA, an amplicon, or combinations thereof, comprise a reporter group. In certain embodiments, a reporter group emits a fluorescent, a chemiluminescent, a bioluminescent, a phosphorescent, or an electrochemiluminescent signal. Some non-limiting examples of reporter groups include fluorophores, radioisotopes, chromogens, enzymes, antigens including epitope tags, semiconductor nanocrystals such as quantum dots, heavy metals, dyes, phosphorescence groups, chemiluminescent groups, electrochemical detection moieties, binding proteins, phosphors, rare earth chelates, transition metal chelates, near-infrared dyes, electrochemiluminescence labels, and mass spectrometer-compatible reporter groups, such as mass tags, charge tags, and isotopes (see, e.g., Haff and Smirnov, Nucl. Acids Res. 25:3749-50, 1997; Xu et al., Anal. Chem. 69:3595-3602, 1997; Sauer et al., Nucl. Acids Res. 31:e63, 2003). “Quantum dots” refer to semiconductor nanocrystalline compounds capable of emitting a second energy in response to exposure to a first energy. Typically, the energy emitted by a single quantum dot has the same predictable wavelength. Exemplary semiconductor nanocrystalline compounds include, but are not limited to, crystals of CdSe, CdS, and ZnS. Descriptions of quantum dots can be found in, among other places, U.S. Pat. Nos. 5,990,479 and 6,207,392 B1 and Han et al., Nature Biotechnology, 19:631-635, 2001.

The term reporter group also encompasses an element of multi-element reporter systems, including, affinity tags such as biotin:avidin, antibody:antigen, and the like, in which one element interacts with one or more other elements of the system in order to affect the potential for a detectable signal. Some non-limiting examples of multi-element reporter systems include an oligonucleotide comprising a biotin reporter group and a streptavidin-conjugated fluorophore, or vice versa; an oligonucleotide comprising a DNP reporter group and a fluorophore-labeled anti-DNP antibody; and the like. Detailed protocols for attaching reporter groups to nucleic acids can be found in, among other places, Hermanson, Bioconjugate Techniques, Academic Press, San Diego, 1996; Beaucage et al.; and Haugland.

Multi-element interacting reporter groups are also within the intended scope of the term reporter group, such as fluorophore-quencher pairs, including fluorescent quenchers and dark quenchers (also known as non-fluorescent quenchers). A fluorescent quencher can absorb the fluorescent signal emitted from a fluorescent reporter group and after absorbing enough fluorescent energy, the fluorescent quencher can emit fluorescence at a characteristic wavelength, e.g., fluorescent resonance energy transfer (FRET). For example without limitation, the FAM-TAMRA pair can be illuminated at 492 nm, the excitation peak for FAM, and emit fluorescence at 580 nm, the emission peak for TAMRA. A dark quencher, appropriately paired with a fluorescent reporter group, absorbs the fluorescent energy from the fluorophore, but does not itself fluoresce. Rather, the dark quencher dissipates the absorbed energy, typically as heat. Some non-limiting examples of dark or nonfluorescent quenchers include Dabcyl, Black Hole Quenchers, Iowa Black, QSY-7, AbsoluteQuencher, Eclipse non-fluorescent quencher, metal clusters such as gold nanoparticles, and the like. Certain dual-labeled probes comprising fluorescent reporter group-quencher pairs can emit fluorescence when the members of the pair are physically separated, for example but without limitation, nuclease probes such as TaqMan® probes. Other dual-labeled probes comprising fluorescent reporter group-quencher pairs can emit fluorescence when the members of the pair are spatially separated, for example but not limited to hybridization probes such as molecular beacons or extension probes such as Scorpion primers. Fluorophore-quencher pairs are well known in the art and used extensively for a variety of reporter probes (see, e.g., Yeung et al., BioTechniques 36:266-75, 2004; Dubertret et al., Nat. Biotech. 19:365-70, 2001; and Tyagi et al., Nat. Biotech. 18:1191-96, 2000).

In some embodiments, a primer and/or an amplification product comprise an affinity tag. In some embodiments, an affinity tag comprises a reporter group. In certain embodiments, affinity tags are used for separating, are part of a detecting means, or both.

In some embodiments, a primer and/or an amplification product comprises a hybridization tag, a hybridization tag complement, or both. In certain embodiments, the same hybridization tag is used with a multiplicity of different elements to effect bulk separation and/or attachment to a solid surface, for example but not limited to certain hybridization-based pullout formats (see, e.g., ABI PRISM® Duplex™ 384 Well F/R Sequence Capture Kit, Applied Biosystems). In various embodiments, hybridization tag complements serve as capture moieties for attaching a hybridization tag:element complex to a solid support, for example but not limited to a particular address or location on a microarray or bead array; serve as “pull-out” sequences for bulk separation procedures or hybridization-based pullout; or both as capture moieties and as pull-out sequences. In certain embodiments, a hybridization tag complement comprises a reporter group, a mobility modifier, a reporter probe-binding portion (for example but not limited to, a sequence that selectively hybridizes with a TaqMan® probe or other nuclease probe, a molecular beacon probe or other hybridization probe, a scorpion primer or other extension primer, and so forth), or combinations thereof.

Typically, hybridization tags and their corresponding hybridization tag complements are selected to minimize internal self-hybridization or cross-hybridization with different hybridization tag species, nucleotide sequences in an amplification composition, including gDNA, different species of hybridization tag complements, primers, primer-binding sites or promoter sequences of amplification products, and the like; but should be amenable to facile hybridization between the hybridization tag and its corresponding hybridization tag complement. In some embodiments, however, a primer-binding site of an amplification product, or at least part of these sequences, can serve as a hybridization tag for the amplification product (see, e.g., ABI PRISM® Duplex™ 384 Well F/R Sequence Capture Kit, Applied Biosystems). Hybridization tag sequences and hybridization tag complement sequences can be selected by any suitable method, for example but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65, 1998). Descriptions of hybridization tags, hybridization tag complements, and their use can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein); Gerry et al., J. Mol. Biol. 292:251-262, 1999) (referred to as “zip-codes” and “zip-code complements” therein); and Brenner et al., Proc. Natl. Acad. Sci. 97:1665-70, 2000 (referred to as “oligonucleotide tags”, “tags”, and “anti-tags” therein). Those in the art will appreciate that a hybridization tag and its corresponding hybridization tag complement are, by definition, complementary to each other and that the terms hybridization tag and hybridization tag complement are relative and can essentially be used interchangeably in most contexts.

Hybridization tags can be located at or near the end of a primer and/or an amplification product; or they can be located internally. In certain embodiments, a hybridization tag is attached to a primer and/or an amplification product via a linker arm. In certain embodiments, the linker arm is cleavable.

In certain embodiments, hybridization tags are at least 12 bases in length, at least 15 bases in length, 12-60 bases in length, or 15-30 bases in length. In certain embodiments, a hybridization tag is 12, 15, 20, 21, 22, 23, 24, 25, 26, 27, 28, 29, 30, 45, or 60 bases in length. In certain embodiments, at least two hybridization tag:hybridization tag complement duplexes have melting temperatures that fall within a Δ Tₘ range (Tₘₐₓ-Tₘᵢₙ) of no more than 10° C. of each other. In certain embodiments, at least two hybridization tag:hybridization tag complement duplexes have melting temperatures that fall within a Δ Tₘ range of 5° C. or less of each other.

In some embodiments, at least one of: a primer, a MSA, and an amplification product comprise a mobility modifier. In certain embodiments, mobility modifiers comprise nucleotides of different lengths effecting different mobilities. In certain embodiments, mobility modifiers comprise non-nucleotide polymers, for example but not limited to, polyethylene oxide (PEO), polyglycolic acid, polyurethane polymers, polypeptides, and oligosaccharides. In certain embodiments, mobility modifiers may work by adding size to a polynucleotide, or by increasing the “drag” of the molecule during migration through a medium without substantially adding to the size. Certain mobility modifiers, including PEO's, have been described in, among other places, U.S. Pat. Nos. 5,470,705; 5,580,732; 5,624,800; and 5,989,871 and United States Patent Application Publication No. US 2003/0190646 A1.

Certain embodiments of the disclosed methods and kits comprise a microfluidics device for at least one of: sample preparation; an amplification reaction; a purifying step; and analyzing at least part of an amplification product. A microfluidics device is a reaction vessel comprising at least one microchannel, generally comprising an internal dimension of one millimeter or less. Microfluidics device typically employ very small reaction volumes, often on the order of one or a few microliters, nanoliters (nL), or picoliters (pL). Those in the art will appreciate that the size, shape, and composition of a microfluidics device is generally not a limitation of the current teachings. Rather, a variety of suitable microfluidics devices can be employed in performing one or more steps of the disclosed methods. Descriptions of exemplary microfluidics devices and uses thereof can be found in, among other places, Fiorini and Chiu, BioTechniques 38:429-46, 2005; Kelly and Woolley, Analyt. Chem. 77(5):96A-102A, 2005; Cheuk-Wai Kan et al., Electrophoresis 25:3564-88, 2004; and Yeun et al., Genome Res. 11:405-12, 2001.

The term “reporter probe” refers to a sequence of nucleotides, nucleotide analogs, or nucleotides and nucleotide analogs, that binds to or anneals with an amplicon, and when detected, including a change in intensity or of emitted wavelength, is used to identify and/or quantify the corresponding amplicon. Reporter probes are typically employed in real-time and certain end-point detection techniques. Most reporter probes can be categorized based on their mode of action, for example but not limited to: nuclease probes, including TaqMan® probes (see, e.g., Livak, Genetic Analysis: Biomolecular Engineering 14:143-149, 1999; Yeung et al., BioTechniques 36:266-75, 2004); extension probes such as scorpion primers, Lux™ primers, Amplifluors, and the like; hybridization probes such as molecular beacons, Eclipse probes, light-up probes, pairs of singly-labeled reporter probes, hybridization probe pairs, and the like; or combinations thereof. In certain embodiments, reporter probes comprise an amide bond, an LNA, a universal base, or combinations thereof, and include stem-loop and stem-less reporter probe configurations. Certain reporter probes are singly-labeled, while other reporter probes are doubly-labeled. Dual probe systems that comprise FRET between adjacently hybridized probes or that collectively comprise a fluor-quencher pair are within the intended scope of the term reporter probe.

In certain embodiments, a reporter probe comprises a fluorescent reporter group, a quencher reporter group (including dark quenchers and fluorescent quenchers), an affinity tag, a hybridization tag, a hybridization tag complement, or combinations thereof. In certain embodiments, a reporter probe comprising a hybridization tag complement anneals with the corresponding hybridization tag, a member of a multi-component reporter group binds to a reporter probe comprising the corresponding member of the multi-component reporter group, or combinations thereof. Exemplary reporter probes include TaqMan® probes; Scorpion probes (also referred to as scorpion primers); Lux™ primers; FRET primers; Eclipse probes; molecular beacons, including FRET-based molecular beacons, multicolor molecular beacons, aptamer beacons, PNA beacons, and antibody beacons; reporter group-labeled PNA clamps, reporter group-labeled PNA openers, reporter group-labeled LNA probes, and probes comprising nanocrystals, metallic nanoparticles and similar hybrid probes (see, e.g., Dubertret et al., Nature Biotech. 19:365-70, 2001; Zelphati et al., BioTechniques 28:304-15, 2000). In certain embodiments, reporter probes further comprise groove binders including TaqMan®MGB probes and TaqMan®MGB-NFQ probes (both from Applied Biosystems). In certain embodiments, reporter probe detection comprises fluorescence polarization detection (see, e.g., Simeonov and Nikiforov, Nucl. Acids Res. 30:e91, 2002).

Certain Exemplary Component Techniques

According to the instant teachings, gDNA may be obtained from any living, or once living, organism, including a prokaryote, an archaea, or a eukaryote, for example but not limited to, an insect including Drosophila, a worm including C. elegans, a plant, and an animal, including a human; and including prokaryotic cells and cells, tissues, and organs obtained from a eukaryote, for example but not limited to, cultured cells and blood cells. Certain viral genomic DNA is also within the scope of the current teachings. In certain embodiments, the gDNA may be present in a double-stranded or single-stranded form. The skilled artisan appreciates that gDNA includes not only full length material, but also fragments generated by any number of means, for example but not limited to, enzyme digestion, sonication, shear force, and the like, and that all such material, whether full length or fragmented, represent forms of gDNA that can serve as templates for an amplifying reaction of the current teachings.

A variety of methods are available for obtaining gDNA for use with the current teachings. Methylated and unmethylated gDNA is also commercially available. When the gDNA is obtained through isolation from a biological matrix, preferred isolation techniques include (1) organic extraction followed by ethanol precipitation, e.g., using a phenol/chloroform organic reagent (see, e.g., Sambrook et al.; Ausubel et al.), for example using an automated DNA extractor, e.g., the Model 341 DNA Extractor (Applied Biosystems, Foster City, Calif.); (2) stationary phase adsorption methods (e.g., Boom et al., U.S. Pat. No. 5,234,809; Walsh et al., Biotechniques 10(4): 506-513, 1991); and (3) salt-induced DNA precipitation methods (see, e.g., Miller et al., Nucl. Acids Res. 16(3): 9-10, 1988), such precipitation methods being typically referred to as “salting-out” methods. In certain embodiments, gDNA isolation techniques comprise an enzyme digestion step to help eliminate unwanted protein from the sample, for example but not limited to, digestion with proteinase K, or other like proteases; a detergent; or both (see, e.g., U.S. Patent Application Publication 2002/0177139; and U.S. patent application Ser. Nos. 09/724,613 and 10/618,493). Commercially available nucleic acid extraction systems include, among others, the ABI PRISM® 6100 Nucleic Acid PrepStation and the ABI PRISM® 6700 Nucleic Acid Automated Work Station; nucleic acid sample preparation reagents and kits are also commercially available, including, NucPrep™ Chemistry, BloodPrep™ Chemistry, the ABI PRISM® TransPrep System, and PrepMan™ Ultra Sample Preparation Reagent (all from Applied Biosystems).

The term “mobility-dependent analysis technique” refers to any analysis method based on different rates of migration between different analytes. Non-limiting examples of mobility-dependent analysis techniques include chromatography, sedimentation, gradient centrifugation, field-flow fractionation, multi-stage extraction techniques, mass spectrometry, and electrophoresis, including slab gel, isoelectric focusing, and capillary electrophoresis.

The terms “amplifying” and “amplification” are used in a broad sense and refer to any technique by which a target region, an amplicon, or at least part of an amplicon, is reproduced or copied (including the synthesis of a complementary strand), typically in a template-dependent manner, including a broad range of techniques for amplifying nucleic acid sequences, either linearly or exponentially. Some non-limiting examples of amplification techniques include primer extension, including the polymerase chain reaction (PCR), RT-PCR, asynchronous PCR (A-PCR), and asymmetric PCR, strand displacement amplification (SDA), multiple displacement amplification (MDA), nucleic acid strand-based amplification (NASBA), rolling circle amplification (RCA), transcription-mediated amplification (TMA), and the like, including multiplex versions and/or combinations thereof. Descriptions of certain amplification techniques can be found in, among other places, Molecular Cloning, A Laboratory Manual, Cold Spring Harbor Press, 3d ed., 2001 (hereinafter “Sambrook and Russell”); Sambrook et al.; Ausubel et al.; PCR Primer: A Laboratory Manual, Diffenbach, Ed., Cold Spring Harbor Press (1995); Msuih et al., J. Clin. Micro. 34:501-07 (1996); McPherson; Rapley; U.S. Pat. Nos. 6,027,998 and 6,511,810; PCT Publication Nos. WO 97/31256 and WO 01/92579; Ehrlich et al., Science 252:1643-50 (1991); Favis et al., Nature Biotechnology 18:561-64 (2000); Protocols & Applications Guide, rev. 9/04, Promega, Madison, Wis.; and Rabenau et al., Infection 28:97-102 (2000).

The terms “amplification product” and “amplicon” are essentially used interchangeably herein and refer to the nucleic acid sequences generated from any cycle of amplification of any amplification reaction, for example a first amplicon is generated during a first amplification reaction and a second amplicon product is generated during a second amplification reaction, unless otherwise apparent from the context. An amplicon can be either double-stranded or single-stranded, including the separated component strands obtained from a double-stranded amplification product.

In certain embodiments, amplification techniques comprise at least one cycle of amplification, for example, but not limited to, the steps of: selectively hybridizing a primer to a target region flanking sequence or a primer-binding site of an amplicon (or complements of either, as appropriate); synthesizing a strand of nucleotides in a template-dependent manner using a polymerase; and denaturing the resulting nucleic acid duplex to separate the strands. The cycle may or may not be repeated.

Amplification can comprise thermocycling or can be performed isothermally. In some embodiments, amplifying comprises a thermocycler, for example but not limited to a GeneAmp® PCR System 9700, 9600, 2700, or 2400 thermocycler (all from Applied Biosystems). In some embodiments, double-stranded amplification products are not initially denatured, but are used in their double-stranded form in one or more subsequent steps. In certain embodiments, single-stranded amplicons are generated in an amplification reaction, for example but not limited to asymmetric PCR or A-PCR.

Primer extension according to the present teachings is an amplification process comprising elongating a primer that is annealed to a template in the 5′ to 3′ direction using a template-dependent polymerase. According to certain embodiments, with appropriate buffers, salts, pH, temperature, and appropriate dNTPs (which may, but need not, comprise at least one MSA), a template-dependent polymerase incorporates nucleotides complementary to the template strand starting at the 3′-end of an annealed primer, to generate a complementary strand. In certain embodiments, the polymerase used for primer extension lacks or substantially lacks 5′-exonuclease activity, 3′-exonuclease activity, or both. Descriptions of certain primer extension reactions can be found in, among other places, Sambrook et al., Sambrook and Russell, and Ausubel et al.

In certain embodiments, an amplification reaction comprises multiplex amplification, in which a multiplicity of different target regions, a multiplicity of different amplification product species, or both, are simultaneously amplified using a multiplicity of different primer pairs (see, e.g., Henegariu et al., BioTechniques 23:504-11, 1997; and Rapley, particularly in Chapter 79). Certain embodiments of the disclosed methods comprise a multiplex amplification reaction and a single-plex amplification reaction, including a multiplicity of single-plex reactions performed in parallel.

In certain embodiments, an amplifying reaction comprises asymmetric PCR. According to certain embodiments, asymmetric PCR comprises an amplification composition comprising (i) at least one primer pair in which there is an excess of one primer, relative to the corresponding primer of the primer pair, for example but not limited to a five-fold, a ten-fold, or a twenty-fold excess; (ii) at least one primer pair that comprises only a forward primer or only a reverse primer; (iii) at least one primer pair that, during given amplification conditions, comprises a primer that results in amplification of one strand and a corresponding primer that is disabled; or (iv) at least one primer pair that meets the description of both (i) and (iii) above. Consequently, when the gDNA target region or an amplification product is amplified, an excess of one strand of the subsequent amplification product (relative to its complement) is generated. Descriptions of asymmetric PCR, can be found in, among other places, McPherson, particularly in Chapter 5; and Rapley, particularly in Chapter 64.

In certain embodiments, one may use at least one primer pair wherein the melting temperature (Tm₅₀) of one of the primers is higher than the Tm₅₀ of the other primer, sometimes referred to as A-PCR (see, e.g., Published U.S. Patent Application No. US 2003-0207266 A1). In certain embodiments, the TM₅₀ of the forward primer is at least 4-15° C. different from the Tm₅₀ of the corresponding reverse primer. In certain embodiments, the Tm₅₀ of the forward primer is at least 8-15° C. different from the Tm₅₀ of the corresponding reverse primer. In certain embodiments, the Tm₅₀ of the forward primer is at least 10-15° C. different from the Tm₅₀ of the corresponding reverse primer. In certain embodiments, the Tm₅₀ of the forward primer is at least 10-12° C. different from the Tm₅₀ of the corresponding reverse primer. In certain embodiments, in at least one primer pair, the Tm₅₀ of a forward primer differs from the Tm₅₀ of the corresponding reverse primer by at least about 4° C., by at least about 8° C., by at least about 10° C., or by at least about 12° C.

In certain embodiments of A-PCR, in addition to the difference in Tm₅₀ of the primers in a primer pair, there is also an excess of one primer relative to the other primer in the primer pair. In certain embodiments, there is a five- to twenty-fold excess of one primer relative to the other primer in the primer pair. In certain embodiments of A-PCR, the primer concentration is at least 50 nM.

In A-PCR according to certain embodiments, one may use conventional PCR in the first cycles of amplification such that both primers anneal and both strands of a double-stranded amplicon or gDNA are amplified. By raising the temperature in subsequent cycles of the same amplification reaction, however, one may disable the primer with the lower Tₘ such that only one strand is amplified. Thus, the subsequent cycles of A-PCR in which the primer with the lower Tₘ is disabled result in asymmetric amplification. Consequently, when the target region or an amplification product is amplified, an excess of one strand of the subsequent amplification product (relative to its complement) is generated.

According to certain embodiments of A-PCR, the level of amplification can be controlled by changing the number of cycles during the first phase of conventional PCR cycling. In such embodiments, by changing the number of initial conventional cycles, one may vary the amount of the double-stranded amplification products that are subjected to the subsequent cycles of PCR at the higher temperature in which the primer with the lower Tₘ is disabled.

Certain methods of optimizing amplification reactions are known to those skilled in the art. For example, it is known that PCR may be optimized by altering times and temperatures for annealing, polymerization, and denaturing, as well as changing the buffers, salts, and other reagents in the reaction composition. Optimization may also be affected by the design of the primers used. For example, the length of the primers, as well as the G-C:A-T ratio may alter the efficiency of primer annealing, thus altering the amplification reaction. Descriptions of amplification optimization can be found in, among other places, James G. Wetmur, “Nucleic Acid Hybrids, Formation and Structure,” in Molecular Biology and Biotechnology, pp. 605-8, (Robert A. Meyers ed., 1995); McPherson, particularly in Chapter 4; Rapley; and Protocols & Applications Guide, rev. 9/04, Promega.

Certain amplification compositions comprise dUTP and uracil-N-glucosidase (UNG). Discussion of use of dUTP and UNG may be found, for example, in Kwok et al., Nature, 339:237-238, 1989; and Longo et al., Gene, 93:125-128, 1990.

In some embodiments, an amplification reaction is followed by a “clean-up” or “purifying” step, wherein at least some of the components of the amplification composition are removed from at least some of the amplicons, thereby purifying the amplicons. Purifying typically comprises a degrading means, including an enzyme such as a nuclease or a phosphatase, or a separating means, including a physical separation means such as a spin column or a separation based on hybridization, such as hybridization-based pullout. For example but not limited to, degrading and/or separating at least some of the unincorporated primers, unincorporated NTPs, enzymes including a polymerase, salts, other amplification composition components, or combinations thereof. In some embodiments, purifying an amplification product comprises a “spin column” or other centrifugal or gel-based separation means; a degradation reaction comprising for example an exonuclease, a phosphatase, or both (e.g., ExoSAP-It® reagent. USB Corp. Cleveland, Ohio), or an exonuclease and an apyrase; a hybridization-based separation means; or a precipitation step, for example but not limited to, ethanol precipitation in the presence of a salt, such as sodium or potassium acetate. Those in the art will appreciate that in certain embodiments, purifying an amplification product can, among other things, decrease the amount of primers needed in a subsequent amplification reaction, decrease possible side reactions, and/or reduce competition due to unincorporated primers and/or dNTPs from a previous amplification reaction.

The term “degrading” is used in a broad sense herein and refers to any technique in which an unincorporated dNTP or nucleotide analog is rendered unincorporable, typically by enzymatic digestion by a phosphatase; an unincorporated primer is digested, typically by an nuclease; or both.

In some embodiments, purifying comprises a nuclease, such as a DNase, for example but not limited to exonuclease I, mung bean nuclease, S1 nuclease, exonuclease T, or combinations thereof. In some embodiments, a dNTP and/or an unincorporated primer is degraded. In some embodiments, unincorporated dNTPs are degraded using an apyrase or a phosphatase, including shrimp alkaline phosphatase (SAP) or calf intestinal phosphatase (CIP). In some embodiments, degrading unincorporated primers and unincorporated dNTPs comprises an apyrase, an inorganic pyrophosphate (PPi), and an exonuclease. Those in the art will appreciate that the method for degrading unincorporated primers and/or unincorporated dNTPs is typically not limiting, provided that the desired polynucleotides, typically amplification products, are not degraded or at least not substantially degraded, while the unincorporated primers and dNTPs are degraded.

In some embodiments, unincorporated primers, unincorporated dNTPs, amplification composition reagents, or combinations thereof, are separated from an amplification product by, for example but not limited to, gel or column purification, sedimentation, filtration, beads, including streptavidin-coated beads, magnetic separation, or hybridization-based pull out, including annealing amplification products comprising hybridization tags to a solid support. A number of kits and reagents for performing such separation techniques are commercially available, including the Wizard® MagneSil™ PCR Clean-Up System (Promega), the MinElute PCR Purification Kit, the QIAquick Gel Extraction Kit, the QIAquick Nucleotide Removal Kit, the QIAquick 96 PCR Purification Kit or BioRobot Kit (all from Qiagen, Valencia, Calif.), Dynabeads® (Dynal Biotech), or the ABI PRISM™ Duplex™ 384 Well F/R Sequence Capture Kit (Applied Biosystems P/N 4308082). In some embodiments, an amplification product is not purified prior to a subsequent amplifying reaction.

In certain embodiments, the disclosed methods and kits comprise a solid support. Non-limiting examples of solid supports include, agarose, sepharose, polystyrene, polyacrylamide, glass, membranes, silica, semiconductor materials, silicon, organic polymers; optically identifiable micro-cylinders; biosensors comprising transducers; appropriately treated or coated reaction vessels and surfaces, for example but not limited to, micro centrifuge or reaction tubes, wells of a multiwell microplate, and glass, quartz or plastic slides and/or cover slips; and beads, for example but not limited to magnetic beads, paramagnetic beads, polymer beads, metallic beads, dye-impregnated or labeled beads, coated beads, glass beads, microspheres and nanospheres. In some embodiments, a solid support is used in a separating and/or detecting step, for example but not limited to, for purifying and/or analyzing amplification products. Those in the art will appreciate that any number of solid supports may be employed in the disclosed methods and kits and that the shape and composition of the solid support is generally not limiting.

In some embodiments, the methods of the current teachings are performed before, after, or in conjunction with a Q-PCR reaction. The term “quantitative PCR”, or “Q-PCR”, refers to a variety of methods used to quantify the results of the polymerase chain reaction for specific nucleic acid sequences. Such methods typically are categorized as kinetics-based systems, that generally determine or compare the amplification factor, such as determining the threshold cycle (C₁), or as co-amplification methods, that generally compare the amount of product generated from simultaneous amplification of target and standard templates. Many Q-PCR techniques comprise reporter probes, intercalating agents, or both. For example but not limited to TaqMan® probes (Applied Biosystems), i-probes, molecular beacons, Eclipse probes, scorpion primers, Lux™ primers, FRET primers, ethidium bromide, SYBR® Green I (Molecular Probes), and PicoGreen® (Molecular Probes).

In some embodiments, the methods of the current teachings are performed before, after, or in conjunction with a sequencing reaction. The term “sequencing” is used in a broad sense herein and refers to any technique known in the art that allows the order of at least some consecutive nucleotides in at least part of a polynucleotide to be identified. Some non-limiting examples of sequencing techniques include Sanger's dideoxy terminator method and the chemical cleavage method of Maxam and Gilbert, including variations of those methods; sequencing by hybridization; and restriction mapping. Some sequencing methods comprise electrophoresis, including capillary electrophoresis and gel electrophoresis; sequencing by hybridization including microarray hybridization; mass spectrometry; and single molecule detection. In some embodiments, sequencing comprises direct sequencing, duplex sequencing, cycle sequencing, single base extension sequencing (SBE), solid-phase sequencing, or combinations thereof. In some embodiments, sequencing comprises detecting the sequencing product using an instrument, for example but not limited to an ABI PRISM® 377 DNA Sequencer, an ABI PRISM® 310, 3100, 3100-Avant, 3730, or 3730xl Genetic Analyzer, an ABI PRISM® 3700 DNA Analyzer (all from Applied Biosystems), or a mass spectrometer. In some embodiments, sequencing comprises incorporating a dNTP, including a dATP, a dCTP, a dGTP, a dTTP, a dUTP, a dITP, or combinations thereof and including dideoxyribonucleotide versions of dNTPs, into an amplification product.

Those in the art will appreciate that the sequencing method employed is not typically a limitation of the present methods. Rather any sequencing technique that provides the order of at least some consecutive nucleotides of at least part of the corresponding extension product or at least part of a vector insert derived from an extension product can typically be used with the current methods. Descriptions of sequencing techniques can be found in, among other places, McPherson, particularly in Chapter 5; Sambrook and Russell; Ausubel et al.; Siuzdak, The Expanding Role of Mass Spectrometry in Biotechnology, MCC Press, 2003, particularly in Chapter 7; and Rapley.

The term “analyzing” when used in reference to a first amplicon, part of a first amplicon, a second amplicon, part of a second amplicon, or combinations thereof, includes any technique that allows one or more parameter of an amplicon or at least part of an amplicon to be obtained. In certain embodiments, analyzing comprises (1) separating (at least partially) one amplicon species from another amplicon species, including amplicons derived from different target regions and amplicons derived from the same target region but with different degrees of methylation (e.g., fully methylated, unmethylated, and intermediate levels of methylation, sometimes referred to as a group or family of “related amplicons”), (2) detecting a separated and/or partially separated amplicon, and (3) obtaining and evaluating one or more amplicon parameter, for example but not limited to, amplicon peak height, integrated area under an amplicon peak, and amplicon intensity, including the fluorescent intensity of an incorporated fluorescent reporter group, the luminescent intensity of an incorporated bioluminescent, chemiluminescent, and/or phosphorescent reporter group, and the radioactive intensity of an incorporated isotope. Typically, one or more parameter(s) of one amplicon is compared with the same parameter(s) of another amplicon to determine the degree of target region methylation, including qualitative, semi-quantitative, and quantitative determinations. The degree of methylation of at least one target region is typically determined by inference, for example but not limited to, by determining whether an amplicon derived from a modified sample comprises a modified nucleotide or its complement and inferring that the corresponding target region is methylated or is not methylated.

In some embodiments, the disclosed methods and kits comprise a microfluidics device, “lab on a chip”, or micrototal analytical system (μTAS). In some embodiments, sample preparation is performed using a microfluidics device. In some embodiments, an amplification reaction is performed using a microfluidics device. In some embodiments, a sequencing or Q-PCR reaction is performed using a microfluidic device. In some embodiments, the nucleotide sequence of at least a part of an amplification product is obtained using a microfluidics device. Descriptions of exemplary microfluidic devices can be found in, among other places, Published PCT Application Nos. WO/0185341 and WO 04/011666; Kartalov and Quake, Nucl. Acids Res. 32:2873-79, 2004; and Fiorini and Chiu, BioTechniques 38:429-46, 2005.

Certain Exemplary Embodiments

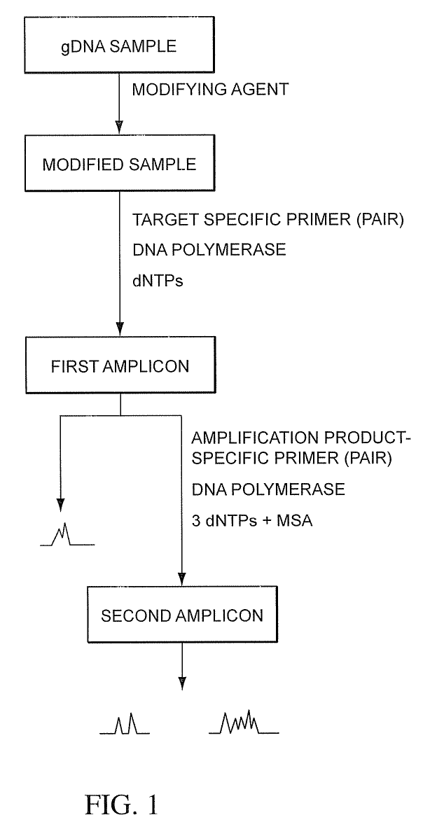

The present teachings provide methods and kits for determining the degree of methylation of at least one target region and for quantitating the number of methylated nucleotides in a given target region, by modifying certain target nucleotides within the target region and then analyzing the amplicon of that modified target region. Although a particular gDNA target region has the same number of nucleotides, whether it comprises some methylated nucleotides or it consists entirely of unmethylated nucleotides, the inventors have discovered that according to the present teachings, after treatment with a modifying agent, the amplicons derived from such methylated, partially methylated, and unmethylated target regions can, under appropriate conditions, be distinguished based on their relative mobilities; and the degree of methylation of the corresponding gDNA target region can be inferred. In some embodiments, at least one MSA is incorporated into certain amplicons, and the amplicons derived from methylated, partially methylated, and unmethylated target regions can be distinguished based on their relative mobilities; and the degree of methylation of the corresponding gDNA target region in the sample can be inferred. In certain embodiments, the number of methylated nucleotides in a gDNA target region can be determined. In some embodiments, the mobility of a double-stranded amplicon or at least part of a double-stranded amplicon is analyzed using a mobility dependent analysis technique. In some embodiments, the mobility of a singe-stranded amplicon or at least part of a single-stranded amplicon is analyzed using a mobility dependent analysis technique. In some embodiments, the mobility dependent analysis technique comprises electrophoresis, for example but not limited to gel electrophoresis and capillary electrophoresis. In some embodiments, at least two amplicons are fully separated so they are detected as at least two individual peaks (see, e.g., FIG. 1, lower left). In some embodiments, two amplicons are not fully separated and are detected as two partially overlapping, but distinguishable peaks (see, e.g., FIG. 1, middle left). According to the present teachings, two overlapping amplicon peaks may be at least partially separated, two partially overlapping amplicon peaks can be fully resolved, or both, by incorporating at least one MSA into at least one amplicon, and/or altering one or more separating conditions, for example but not limited to, buffer composition, pH, temperature, polymer composition and/or concentration, and capillary length.

In certain embodiments, an amplicon is automatically detected by UV absorption or laser-induced fluorescence as the fragment passes through the detector assembly of an instrument, such as a capillary electrophoresis instrument, the detection data is collected and stored in a computer and analyzed by the associated software to determine the fragment's mobility and certain other relevant parameters, for example but not limited to, peak height, area under the peak, fluorescent intensity, and so forth. In certain embodiments, the degree of target region methylation is determined by obtaining the ratio of one or more migration parameters for an amplicon of a fully methylated target region, the corresponding target region with at least some intermediate methylation, and/or the corresponding unmethylated target region. Such an analysis may include a comparison of at least one test amplicon parameter with at least one control amplicon parameter, wherein the test amplicon is derived from the target region being evaluated.

In some embodiments, determining the degree of methylation of at least one target region comprises evaluating an internal standard or a control sequence, such as a standard curve for the corresponding target region, an internal size standard, a fully methylated target region control sequence, a fully unmethylated target region control sequence, a target region control sequence comprising a known intermediate level of methylation, or combinations thereof. In some embodiments, determining the degree of methylation of at least one target region comprises comparing the relative migration rate or other measurable parameter of at least one test amplicon with at least one control sequence amplicon. In some embodiments, a control sequence is employed to account for lane-to-lane, capillary-to-capillary, and/or assay-to-assay variability.

The disclosed methods can serve as “stand-alone” techniques for determining the degree of methylation of one target region or a plurality of different target regions, for example but not limited to a multiplex reaction or series of parallel single-plex reactions. The disclosed methods can also be employed in conjunction with, before, or after other molecular biology methods, for example but not limited to Q-PCR, sequencing, or certain other methylation analysis techniques (see, e.g., Fraga and Esteller, BioTechniques 33:632-49, 2002; DNA Methylation Protocols, Mills and Ramsahoye, eds., Humana Press, 2002; and U.S. Pat. No. 6,331,393). Non-limiting examples of methylation detection and/or quantitation methods that may be employed before, after, and/or in conjunction with certain disclosed methods include melting curve analysis of modified and/or unmodified target regions, methylation specific PCR (MSP), MethyLight, methyl-acceptor assay, enzymatic regional methylation assay (ERMA), certain assays using methylation-sensitive and insensitive restriction endonucleases, for example but not limited to the HpaII/MspI isoschizomer pair, combined bisulfite restriction analysis (COBRA), hydrazine and/or permanganate treatment with or without ligation-mediated PCR, restriction landmark genomic scanning (RLGS), differential methylation hybridization, methylation-specific oligonucleotide microarray technique, methylation assay by nucleotide incorporation (MANIC), methylation-sensitive single nucleotide primer extension (Ms-SnuPE), pyrosequencing methylation analysis (PyroMethA), DHPLC-based DNA methylation analysis; and bisulfite genomic sequencing (see, e.g., Herman et al., Proc. Natl. Acad. Sci. 93:9821-26, 1996; Yamamoto et al., BioTechniques 36(5):846-54, 2004; Eads et al., Nucl. Acids Res. 28(8):e32, 2000; Collela et al., BioTechniques, 35(1):146-50, 2003; Xiong and Laird, Nucl. Acids Res. 25(12):2532-34, 1997; Granau et al., Nucl. Acids Res., 29(13):e65, 2001; Convert et al., BioTechniques 34(2):356-62, 2003; and Kupper et al., BioTechniques 23(5):843-46, 1997).

In some embodiments, an amplification composition comprises a DNA polymerase, a primer pair, a mix of nucleotides comprising dATP, dCTP, dGTP, dTTP, and (i) a MSA of dCTP, (ii) a MSA of dGTP, or (iii) a MSA of dCTP and a MSA of dGTP. In certain such embodiments, the primer pair comprises an unequal molar ratio of forward primers to reverse primers and the amplification reaction comprises multiple cycle asymmetric PCR, wherein the later cycles typically comprise linear amplification. In certain such embodiments, the mix of nucleotides comprises dATP, dCTP, dGTP, dTTP, and a ddNTP, for example but not limited to, a ddCTP comprising a first reporter group, a ddGTP comprising a second reporter group, or a ddCTP comprising a first reporter group and a ddGTP comprising a second reporter group. In certain such embodiments, at least part of the amplification product is analyzed and the degree of methylation of at least one target region is determined.

In certain embodiments, the degree of methylation of a target region in two or more samples is determined in the same or parallel reactions. In some embodiments, the disclosed methods and kits allow the presence of certain secondary amplification products and primer dimer formation to be observed. Such amplification artifacts can result in inaccuracies in some quantitative methods but may go undetected in those methods.

According to the disclosed methods, a sample comprising at least one gDNA target region is exposed to a modifying agent and a modified sample is obtained. The term “modified sample” refers to a sample that has been exposed to a modifying agent under conditions suitable for the modifying agent to generate at least one modified nucleotide. Typically the modifying agent will interact with or convert at least one target nucleotide in the gDNA to generate at least one modified nucleotide. Non-limiting examples of compounds that may serve as suitable modifying agents include bisulfite compounds, for example but not limited to, sodium bisulfite, magnesium bisulfite, manganese bisulfite, potassium bisulfite, ammonium bisulfite; 5-bromouracil; and certain sulfhydryl compounds, for example but not limited to, mercaptoethanol, cysteine methyl ester, glutathione, and cysteamine. Descriptions of exemplary modifying agents can be found in, among other places, Hayatsu, Prog. Nucl. Acid Res. Mol. Biol. 16:75-124, 1975; Hayatsu, Proc. Japanese Acad. Ser. B, 80:189-94, 2004; Boyd and Zon, Anal. Biochem. 326:278-80, 2004; U.S. patent application Ser. No. 10/926,530; and U.S. Published Patent Appl. No. US 2005-008989A1).

In certain embodiments, a sample comprising gDNA is treated with the modifying agent sodium bisulfite, which converts unmethylated cytosines (“target nucleotides”) to uracil (the “modified nucleotide”), while methylated cytosines (also target nucleotides) are generally non-reactive. At least one target region in the bisulfite treated gDNA is amplified, typically by PCR using target-specific primers to yield first amplicons in which uracil residues are converted to thymine, while methylated cytosine is amplified as cytosine. In the case of samples comprising mixed cell populations, for example but not limited to tumor biopsy samples containing both normal cells and cancerous cells, the cytosine content of the amplified DNA from the various cell subpopulations can be very different, with unmethylated DNA being T-rich and C-deficient after conversion, while the amplicons from methylated target regions can retain at least some of the original cytosine content.

In certain embodiments, two or more different samples are individually exposed to a modifying agent in separate reactions to obtain two or more different modified samples. In some embodiments, two or more different modified samples are amplified in parallel reactions according to the disclosed methods, and the degree of methylation of at least one target region in the two or more samples is compared.

According to certain methods, at least one sample comprising or suspected of comprising at least one gDNA target region is exposed to a modifying agent to obtain at least one modified sample that typically comprises at least one modified nucleotide. In certain embodiments, the modifying agent comprises sodium bisulfite and the modified nucleotide comprises a uracil that was derived from an unmethylated cytosine. A first amplification composition is formed comprising: at least some of the modified sample, a target-specific primer pair for each target region to be evaluated, a mixture of appropriate dNTPs, and a first DNA polymerase. The first amplification composition is subjected to at least one cycle of amplification and a first amplification product is generated. According to some methods, the cycle of amplification comprises a multiplicity of cycles of amplification, for example but not limited to, from about 3 to about 45 cycles, expressly including all whole numbers from 3 to 45. The first amplification products are analyzed, typically using a mobility dependent analysis technique, to determine their relative migration rates and the degree of methylation for at least one target region is inferred. In certain embodiments, the migration rate of at least one amplicon is determined using a standard curve or by comparison with a control sequence, for example but not limited to one or more size standards, including one or more nucleic acid fragment of known nucleotide and/or analog composition and length. In some embodiments, analyzing comprises determining the number of modified nucleotides or its complement that are present in a first amplicon or at least one strand of a first amplicon comprising at least part of the sequence of a modified target region or its complement.

In some embodiments, a first amplification composition is formed, comprising at least some modified sample, a target-specific primer pair, a DNA polymerase, and a mix of deoxyribonucleoside triphosphates (dNTPs), as shown in FIG. 1. The target-specific primer pair comprises (1) a forward target-specific primer comprising (a) a first target-specific portion that comprises a sequence that is the same as or substantially the same as, and is designed to selectively hybridize with, the complement of the upstream target flanking sequence and optionally, (b) a tail portion that comprises a primer-binding site, located upstream from the first target-specific portion; and (2) a reverse target-specific primer comprising (a) a second target-specific portion that comprises a sequence that is complementary to or substantially complementary to, and is designed to selectively hybridize with, the downstream target flanking sequence and optionally, (b) a tail portion that comprises a primer-binding site, located upstream from the second target-specific portion. The first reaction composition is subjected to at least one cycle of amplification, typically comprising the sequential steps of template denaturation, primer annealing, and primer extension, and a double-stranded first amplicon is generated, as shown in FIG. 1. In some embodiments, an amplification reaction comprises asymmetric PCR or A-PCR and at least some single-stranded first amplicons are generated. In certain embodiments, both single-stranded and double-stranded amplicons are generated during an amplification reaction.

In some embodiments, at least part of a first amplicon, at least part of a second amplicon, or both are analyzed, typically using a mobility dependent analysis technique. In some embodiments, the analyzing comprises gel electrophoresis on a horizontal or vertical slab gel and the migration rate or other measurable parameter of the amplicon or amplicon fragment is determined. In certain embodiments, the analyzing comprises capillary electrophoresis and the migration rate or other measurable parameter of the amplicon or a fragment of an amplicon is determined. In some embodiments, the amplicon or amplicon fragment is co-electrophoresed with at least one control sequence, for example but not limited to, a molecular size marker or an amplicon or a set of amplicons from the same target region but with known degrees of methylation. In some embodiments, the relative peak height, the area under the peak (curve), or another measurable parameter of the amplicon is determined, typically by a computer and associated software to evaluate the degree of methylation of at least one target region. In some embodiments, the relative peak height or the area under the peak for a given amplicon, or at least part of an amplicon, is compared with a standard curve to evaluate the degree of methylation of at least one target region.

For illustration purposes but not as a limitation, an illustrative biopsy specimen containing a mixed cell population is obtained from a cancer patient to determine the degree of methylation of target region A, among other things. Exemplary target region A contains 5 CG pairs and the gDNA in the specimen contains some copies of target region A that are fully methylated (i.e., all 5 CG pairs comprise 5 mC), some copies containing 2 CG pairs and 3 5 mCG pairs (partially methylated), and some copies that are unmethylated (no 5 mCG). The gDNA is extracted from the specimen and the sample is exposed to a sodium bisulfite to generate a modified sample. Target region A is amplified by PCR in a first amplification composition that comprises a target region A-specific primer pair, wherein the forward target region A-specific primer comprises a fluorescent reporter group, a DNA polymerase, and a mix of dNTPs. The forward strand of the amplicons derived from the fully methylated target region A comprise 5 Cs, the forward strand of the amplicons derived from the partially methylated copies of target region A comprise 3 Cs, and the forward strand of the amplicons derived from the unmethylated copies of target region A do not contain any Cs (excluding any Cs that may be present in the flanking sequences and/or primer tails). These illustrative first amplicons are analyzed by capillary electrophoresis along with an appropriate DNA size ladder and three different, partially overlapping amplicon peaks are detected, indicating that a fully methylated, at least one partially methylated, and an unmethylated species of target region A are present in the sample.

According to certain methods, at least one sample comprising or suspected of comprising at least one gDNA target region is exposed to a modifying agent to generate at least one modified sample comprising at least one target region comprising at least one modified nucleotide. A first amplification composition is formed comprising: at least some of the modified sample, a target-specific primer pair for each target region to be evaluated, a mixture of appropriate dNTPs including at least one MSA, and a first DNA polymerase. In some embodiments, the forward primer, the reverse primer, or the forward and the reverse primer of at least one target-specific primer pair comprises a reporter group, for example but not limited to a fluorescent reporter group. The first amplification composition is subjected to at least one cycle of amplification and a first amplification product comprising at least one incorporated MSA is generated. The incorporation of at least one MSA into an amplification product alters the mobility of that amplification product relative to an amplification product with the same sequence except for the MSA, for example but not limited to, an amplicon comprising the four natural nucleotides. In certain embodiments, the cycle of amplification is repeated for a multiplicity of cycles, for example but not limited to, from about 3 to about 45 cycles, expressly including all whole numbers from 3 to 45. The first amplification products comprising the at least one incorporated MSA are analyzed to determine their relative migration rates or other measurable amplicon parameters and the degree of methylation for at least one target region is inferred. In some embodiments, analyzing comprises determining the number of modified nucleotides or its complement that are present in a first amplicon or at least one strand of a double-stranded first amplicon comprising at least a part of the sequence of a modified target region or its complement.

In certain embodiments, a first amplification composition is formed comprising at least some bisulfite-modified sample, a target-specific primer pair, a polymerase, and a mixture of appropriate dNTPs comprising a MSA. In certain embodiments, the mixture of appropriate dNTPs including at least one MSA comprises dATP, dCTP, dTTP, and either (i) a mobility shifting analog of dCTP, (ii) a mobility shifting analog of dGTP, or (iii) a MSA of dCTP and a MSA of dGTP. The target specific primer pair comprises (1) a forward target-specific primer comprising a first target-specific portion and (2) a reverse target-specific primer comprising a second target-specific portion. In some embodiments, the forward primer, the reverse primer, or both, further comprises a reporter group, typically but not always located at or near the 5′-end of the primer. The first amplification composition is subjected to a multiplicity of cycles of amplification, for example but not limited to, 5 cycles, 10 cycles, 20 cycles, 25 cycles, 30 cycles, or 35 cycles of amplification and a first amplicon comprising a mobility shifting dCTP analog and/or a mobility shifting dGTP analog is generated. Because the sample is modified by sodium bisulfite treatment, the unmethylated Cs in the target region should be deaminated to U, while the methylated Cs in the target region are not modified. When the modified sample is amplified in the amplification composition comprising a mobility shifting analog of dCTP, the reverse target-specific primer anneals with the downstream target flanking region and is extended by the polymerase to generate a first synthesized strand, which serves as a template for the forward target-specific primer. The annealed first primer is extended on the first strand template and the mobility shifting dCTP analog is incorporated into the forward strand of the amplicon where the target sequence comprises the unmodified methylated cytosine, but T is incorporated where the target sequence comprises the modified nucleotide, deaminated C. Thus, when the reporter group labeled amplicon is analyzed, its migration rate should be altered due to the presence of the MSA. In some embodiments, the presence of the MSAs allows the amplicon of a methylated target sequence to be more easily distinguished from the amplicon of the same target sequence that is unmethylated. In certain embodiments, the number of methylated nucleotides present in the target region can be determined by the incremental change in the migration rate of the amplicon relative to the amplicon generated from a modified unmethylated target region, a control sequence of known methylation, or both.

According to certain methods, at least one sample comprising or suspected of comprising at least one gDNA target region is exposed to a modifying agent to generate at least one modified sample comprising at least one target region comprising at least one modified nucleotide. A first amplification composition is formed comprising: at least some of the modified sample, a target-specific primer pair for each target region to be evaluated, a mix of dNTPs, and a first DNA polymerase. The first amplification composition is subjected to at least one cycle of amplification and a first amplification product is generated. According to some methods, the first cycle of amplification comprises a multiplicity of cycles of amplification, for example but not limited to, from about 3 to about 45 cycles, expressly including all whole numbers from 3 to 45. A second amplification composition is formed comprising at least some of the first amplification product, an amplification product primer pair or at least one amplification primer (shown as “Amplification product-Specific Primer (Pair)” in FIG. 1), a mix of appropriate dNTPs including a MSA, and a second DNA polymerase. In some embodiments, the second amplification composition comprises an amplification product primer pair comprising a forward amplification product primer and a reverse amplification product primer. In certain embodiments, the mixture of appropriate dNTPs including at least one MSA comprises dATP, dGTP, dTTP, and either (i) a mobility shifting analog of dCTP, (ii) a mobility shifting analog of dGTP, or (iii) both. The second amplification composition is subjected to at least one cycle of amplification to generate a second amplicon comprising at least one incorporated MSA. According to some methods, the second cycle of amplification comprises a multiplicity of amplification cycles, for example but not limited to, from 2 to about 45 cycles, expressly including all whole numbers from 2 to 45. At least part of the second amplification products are analyzed to determine their relative migration rates or other measurable amplicon parameters and the degree of methylation for at least one target region is inferred. In some embodiments, analyzing comprises determining the number of modified nucleotides or its complement that are present in a second amplicon or at least one strand of a second amplicon comprising at least a part of the sequence of a modified target region or its complement.

In certain embodiments, a first amplification composition is formed comprising at least some bisulfite-modified sample, a multiplicity of different primer pairs, a DNA polymerase, and a mix of dATP, dCTP, dGTP, and dTTP. Each of the target-specific primer pairs comprise (1) a forward target-specific primer comprising (a) a first target-specific portion that comprises a sequence that is the same as or substantially the same as, and is designed to selectively hybridize with the complement of, the upstream target flanking sequence and (b) a tail portion comprising a first primer-binding site that is located upstream from the first target-specific portion; and (2) a reverse target-specific primer comprising (a) a second target-specific portion that comprises a sequence that is complementary to or substantially complementary to, and is designed to selectively hybridize with, the downstream target flanking sequence and (b) a tail portion comprising a second primer-binding site that is located upstream from the second target-specific portion. The first primer-binding site and second primer-binding site of each of the different target-specific primer pairs is different from the first and second primer-binding sites of each of the other different target-specific primer pairs. The first amplification composition is subjected to a multiplicity of cycles of amplification, for example but not limited to 20 cycles, 25 cycles, 30 cycles, 35 cycles, or 40 cycles of amplification, and a multiplicity of different first amplicons are generated. In certain embodiments, the multiplicity of different first amplicons is purified using a separating and/or a degrading means.

A multiplicity of different second amplification compositions are formed, each comprising at least some of the first amplicons or the purified first amplicons, an amplification product primer pair, a DNA polymerase, and a mixture of appropriate dNTPs including at least one MSA. In certain embodiments, the mixture of appropriate dNTPs including at least one MSA comprises dATP, dGTP, dTTP, and either (i) a mobility shifting analog of dCTP, (ii) a mobility shifting analog of dGTP, or (iii) both. In certain embodiments, the mixture of appropriate dNTPs including at least one MSA comprises dATP, dCTP, dTTP, and a mobility shifting analog of dGTP. The amplification product primer pair comprises (1) a forward amplification product primer comprising a sequence that is the same as or substantially the same as the first primer-binding site of a first amplicon and (2) a reverse amplification product primer comprising a sequence that is complementary to or substantially complementary to the second primer-binding site of the same first amplicon. The forward amplification product primer, the reverse amplification product specific primer, or both, further comprises a reporter group. In some embodiments, at least one of the multiplicity of second amplification compositions comprises 2, 3, 4, 5, or 6 different amplification product primer pairs, each comprising a different reporter group than any of the other amplification product primer pairs in the same second amplification composition. In some embodiments, at least some of the multiplicity of second amplification compositions comprise a universal primer or a universal primer pair which, in certain embodiments, comprise the same reporter group, and 2, 3, 4, 5, or 6 differently sized amplicons are generated. The second amplification compositions are subjected to a multiplicity of cycles of amplification, for example but not limited to, 3 cycles, 4 cycles, 5 cycles, 6 cycles, 8 cycles, 10 cycles, or 15 cycles of amplification to generate a second amplicon comprising a MSA in at least one of the second amplification compositions. The second amplicons or at least part of the second amplicons are analyzed, typically using a mobility dependent analysis technique and the degree of methylation for a multiplicity of different target regions is inferred.

For illustration purposes but not as a limitation, an illustrative biopsy specimen containing a mixed cell population is obtained from a cancer patient to determine the degree of methylation of target region B, among other things. Exemplary target region B contains 9 CG pairs and the gDNA in the specimen contains four subpopulations of target region B: some copies of target region B that are fully methylated (i.e., all 9 CG pairs comprise 5 mC), some copies containing 6 CG pairs and 3 5 mCG pairs (partially methylated), some copies containing 3 CG pairs and 6 5 mCG pairs (partially methylated), and some copies that are unmethylated (no 5 mCG). The gDNA is extracted from the specimen and the sample is exposed to a sodium bisulfite to generate a modified sample. Target region B is amplified by PCR in a first amplification composition that comprises a target region B-specific primer pair, a DNA polymerase, and a mix of dNTPs and a family of four related first amplicons is generated. The first amplicons are purified using a spin column and an aliquot of the purified first amplicons is added to a second amplification composition comprising a second DNA polymerase, a first amplicon primer pair, and a mix of dNTPs comprising dATP, dGTP, dTTP, and biotin-36-dCTP, a mobility shifting analog of dCTP. The second amplification composition is subjected to five cycles of amplification to generate second amplicons. The forward strand of the second amplicons derived from the fully methylated target region B comprise 9 biotin-36-dCTPs, the forward strand of the amplicons derived from the partially methylated copies of target region B comprise 3 biotin-36-dCTPs and 6 biotin-36-dCTPs, respectively, and the forward strand of the amplicons derived from the unmethylated copies of target region B do not contain any biotin-36-dCTP (excluding any Cs that may be present in the flanking sequences). The second amplicons are analyzed by capillary electrophoresis along with an appropriate DNA size ladder and four different amplicon peaks are detected based, at least in part, on the incremental mobility shift imparted by the biotin-36-dCTP analog incorporated into three of the four second target region B amplicons. The four amplicon peaks are compared to a standard curve for target region B to determine that the methylation state of target region B in the sample includes some fully methylated target region B, two different subpopulations with intermediate methylation including some with three 5 mCG pairs and some with six 5 mCG pairs, and some unmethylated target region B.

In some embodiments, an amplification composition comprises a nucleotide terminator, also referred to as a terminator, particularly when the amplifying comprises a sequencing reaction for example but not limited to, cycle sequencing or SBE. In certain embodiments, terminators are those in which the nucleotide base is a purine, a 7-deaza-purine, a pyrimidine, or a nucleotide analog, and the sugar moiety is a pentose which includes a 3′-substituent that blocks further synthesis, such as a dideoxynucleoside triphosphate (ddNTP). In certain embodiments, substituents that block further synthesis include, but are not limited to, amino, deoxy, halogen, alkoxy and aryloxy groups. Some non-limiting examples of terminators include, those in which the sugar-phosphate ester moiety is 3′-(C1-C6)alkylribose-5′-triphosphate; 2′-deoxy-3′-(C1-C6)alkylribose-5′-triphosphate; 2′-deoxy-3′-(C1-C6)alkoxyribose 5-triphosphate; 2′-deoxy-3′-(C5-C14)aryloxyribose-5′-triphosphate; 2′-deoxy-3′-haloribose-5′-triphosphate; 2′-deoxy-3′-aminoribose-5′-triphosphate; 2′,3′-dideoxyribose-5′-triphosphate; or 2′,3′-didehydroribose-5′-triphosphate. Terminators also include “T” terminators, including ddTTP and dUTP, which incorporate opposite an adenine, or adenine analog, in a template; “A” terminators, including ddATP, which incorporate opposite a thymine, uracil, or an analog of thymine or uracil, in the template; “C” terminators, including ddCTP, which incorporate opposite a guanine or a guanine analog, in the template; and “G” terminators, including ddGTP and ddITP, which incorporate opposite a cytosine or a cytosine analog, in the template. In some embodiments, a nucleotide terminator comprises a reporter group, for example but not limited to, a fluorescent reporter group.

In some embodiments, the tail portion of a forward target-specific primer, a reverse target-specific primer, a forward amplification product primer, a reverse amplification product primer, or combinations thereof, comprises a universal priming sequence and/or a reporter probe-binding portion. In some embodiments, the universal priming sequence and/or the reporter probe-binding portion is employed in a parallel or subsequent procedure, for example but not limited to, sequencing or quantitative PCR (Q-PCR).

In some embodiments, the first DNA polymerase and the second DNA polymerase are the same. In some embodiments, the first DNA polymerase and the second DNA polymerase are different. In certain embodiments, a first DNA polymerase, a second DNA polymerase, or both, comprises a permissive polymerase that is able to incorporate nucleotide analogs, including MSAs. Non-limiting examples of potentially permissive DNA polymerases include Vent (exo-)® DNA polymerase, Deep Vent® (exo-) DNA polymerase, AmpliTaq DNA polymerase CS, Therminator™ DNA polymerase, Sequenase, bacteriophage T7 DNA polymerase, AmpliTaq DNA polymerase FS, and ThermoSequenase. Those in the art will understand that selection of an appropriate permissive DNA polymerase depends, at least in part, on the analog, the method, and the reaction conditions, but that a suitable permissive polymerase for a particular application can be determined by routine methods and does not require undue experimentation. Those in the art will also understand that a DNA polymerase with desired permissivity can be engineered using well known techniques, including compartmentalized self-replication (CSR) and certain mutagenesis techniques, without undue experimentation (see, e.g., Ghadessy et al., Nature Biotechnol. 22:755-79, 2004; and Singh et al., Protein Engineering 13(9):635-43, 2000).

In some embodiments, the first DNA polymerase is a thermostable DNA polymerase for example but not limited to Taq DNA polymerase or Tli DNA polymerase, including enzymatically active mutants or variants thereof. In some embodiments, the second DNA polymerase is a permissive DNA polymerase. In some embodiments, the first DNA polymerase and the second DNA polymerase are the same. In certain embodiments, there may be sufficient residual first DNA polymerase from the first amplification composition to synthesize the second amplification product and the second amplification composition does not comprise a second DNA polymerase. In certain embodiments, the first DNA polymerase, the second DNA polymerase, or both, comprise a multiplicity of different DNA polymerases. In some embodiments, for example but not limited to embodiments comprising primer concentration-dependent asymmetric PCR, a single DNA polymerase is employed.

The term “analyzing” is used in a broad sense herein and includes any technique or combination of techniques that allow a measurable amplicon parameter to be obtained. In some embodiments, analyzing comprises resolving one or more amplicons using a mobility dependent analysis technique. In some embodiments, a family of related amplicons is analyzed and the degrees of methylation of the target region that corresponds to the group of related amplicons is determined.

In certain embodiments, analyzing comprises separating and/or detecting at least one amplicon or at least part of an amplicon using an instrument, i.e., an automated or semi-automated detection means that can, but need not, comprise a computer algorithm. In certain embodiments, the detection step is combined with or is a continuation of a separating step, for example but not limited to a capillary electrophoresis instrument comprising a fluorescent scanner and a computer that includes a data collection module such as a graphing, recording, or readout component and data analysis software; a capillary electrophoresis instrument coupled with a mass spectrometer; or a chromatography column coupled with an absorbance monitor or fluorescence scanner and a graph recorder, or with a mass spectrometer. Exemplary means for performing an analyzing step include capillary electrophoresis instruments, for example but not limited to, the ABI PRISM® 3100 Genetic Analyzer, ABI PRISM® 3100-Avant Genetic Analyzer, ABI PRISM® 3700 DNA Analyzer, ABI PRISM® 3730 DNA Analyzer, ABI PRISM® 3730xl DNA Analyzer (all from Applied Biosystems); the ABI PRISM® 7300 Real-Time PCR System; the ABI PRISM® 7700 Sequence Detection System; mass spectrometers; and including related software, as appropriate. Exemplary software for reporter group detection, data collection, and analysis includes GeneMapper™ Software, GeneScan® Analysis Software, and Genotyper® software (all from Applied Biosystems).

In certain embodiments, analyzing comprises: separating at least one amplicon and typically two or more amplicons, which may include a family of related amplicons, using a mobility-dependent analytical technique, such as capillary electrophoresis; monitoring the eluate using, for example but without limitation, a fluorescent scanner, to detect the amplicon(s) as they elute; and evaluating the fluorescent profile of the amplicon(s), typically using detection and analysis software, such as an ABI PRISM® Genetic Analyzer using GeneScan® Analysis Software (both from Applied Biosystems) to determine the degree of methylation of at least one target region. In certain embodiments, analyzing comprises a plate reader and an appropriate illumination source.

In certain embodiments, at least two amplicons (for example but not limited to, the amplicons derived from two different target regions and/or at least one amplicon derived from the methylated version of a target region and an amplicon derived from the unmethylated version of the same target region) and/or an amplicon and a control sequence, are resolved and analyzed by electrophoresis in a sieving or non-sieving matrix. In certain embodiments, the electrophoretic separation is carried out in a capillary tube by capillary electrophoresis (see, e.g., Capillary Electrophoresis: Theory and Practice, Grossman and Colburn eds., Academic Press, 1992). Non-limiting examples of sieving matrices for use in the disclosed teachings include covalently crosslinked matrices, such as polyacrylamide covalently crosslinked with bis-acrylamide; gel matrices formed with linear polymers (see, e.g., U.S. Pat. No. 5,552,028); and gel-free sieving media (see, e.g., U.S. Pat. No. 5,624,800; Hubert and Slater, Electrophoresis, 16: 2137-2142, 1995; Mayer et al., Analytical Chemistry, 66(10): 1777-1780, 1994). The electrophoresis medium may contain a nucleic acid denaturant, such as 7M formamide, for maintaining polynucleotides in single stranded form. Suitable capillary electrophoresis instrumentation are commercially available, e.g., the ABI PRISM™ Genetic Analyzer series (Applied Biosystems).

Those in the art will appreciate that the ability to resolve two or more closely migrating fragments, for example but not limited to amplicons and control sequences, is dependent on a number of variables including the resolving power of the analysis technique employed. According to certain of the disclosed methods, the ability to resolve two or more closely migrating amplicons is enhanced by incorporating an MSA into at least one of the amplicons. In certain embodiments, the distance between two closely migrating peaks is increased due to the presence of different numbers of MSAs in the two or more peaks, for example but not limited to an amplicon derived from an unmethylated target region that doesn't contain a MSA and an amplicon derived from a methylated version of the same target region that comprises at least one MSA. In some embodiments, the incorporation of MSAs into an amplicon derived from an unmethylated target region or a family of related amplicons derived from a target region that has multiple subpopulations with differing levels of methylation, can allow the number of methylated nucleotides in that target region to be determined.

Other variables that can affect the separation of two or more closely migrating fragments in an electrophoresis-based analysis technique include the type, concentration, and composition of the sieving matrix; the buffer composition, concentration, and pH; the presence of denaturants or contaminants; the temperature at which the electrophoresis is performed; whether the fragments are single-stranded or double-stranded; voltage applied; electrokinetic inject time; and the length of the capillary or slab gel. The sensitivity of the detection means and analytical software can also affect peak resolution. Those in the art will understand that the separation between two or more closely migrating fragments can be increased or decreased based on one or more of these variables and/or the presence or absence of MSAs in the fragments being analyzed, including the mobility shifting property of the particular MSAs. Those in the art will also understand that appropriate resolving conditions can typically be obtained empirically using routine testing and without undue experimentation.

In certain exemplary embodiments, air-dried amplification product pellets, comprising amplification products, including sequencing reaction products, and/or amplification products of uniquely identifiable molecular weight, are resuspended in water, buffer, or deionized formamide, e.g., HiDi formamide (Applied Biosystems). In certain embodiments, the resuspended samples and a molecular weight marker (e.g., GeneScan 500-LIZ or -ROX size standards, Applied Biosystems) are loaded onto a capillary electrophoresis platform (e.g., ABI PRISM™ Genetic Analyzer, Applied Biosystems) and electrophoresed in an appropriate polymer, for example but not limited to, POP-4, POP-6, or POP-7 polymers (Applied Biosystems). In certain embodiments, the electrophoretic bands comprising at least some of the double-stranded amplicons, single-stranded amplicons, one or both strands of a denatured double-stranded amplicon, or combinations thereof, are detected and their migration rate and/or other amplicon parameters are obtained and evaluated. In certain embodiments, the bands are identified based on their migration rate, peak height, or peak intensity relative to a standard curve or one or more control sequences, and the degree of methylation of at least one corresponding target region is inferred.

In certain embodiments, the degree of target region methylation is determined by (a) comparing one or more measurable amplicon parameters between two or more related amplicon species, for example but not limited to a first amplicon species derived from fully methylated copies of the target region, a second amplicon species derived from unmethylated copies of the target region, and/or one or more amplicon species derived from one or more target regions with intermediate levels of methylation; (b) comparing one or more measurable amplicon parameters with one of more standard curves for the target region; and/or (c) comparing one or more measurable amplicon parameters with a control sequence, for example but not limited to a size ladder.

In certain embodiments, the migration rate or other measurable amplicon parameter is compared to a standard curve for the corresponding target region to determine the degree of methylation of that target region in the sample. Those in the art appreciate that numerous measurable amplicon parameters exist that can be used to compare a group of related amplicons generated from the same target region with differing levels of methylation, including amplicon migration rate, height of the amplicon peak height, integrated area under the curves for the amplicons of the group of related amplicons, and so forth. By evaluating one or more measurable amplicon parameter using a standard curve, a control sequence, or a related amplicon species, one can typically determine the degree of methylation or a target region.

Typically the peak height, the area under the amplicon peak, the signal intensity of one or more detected reporter group on at least one amplicon, or other measurable amplicon parameter are obtained and the degree of methylation of at least one target region can be extrapolated using, for example but without limitation, one or more corresponding standard curves, from which the degree of methylation of the target region that corresponds to the amplicon can be inferred.

Standard curves can be useful for determining the degree of methylation of a particular target region in a sample. The generation and use of standard curves is well known to those in the art (see, e.g., Overholtzer et al., Proc. Natl. Acad. Sci. 100: 11547-52, 2003). Typically, pre-determined mixtures or serial dilutions of synthetic templates or gDNA with known methylation states that comprise one or more target region (“control templates”) are used as the starting material for one or more of the disclosed methods. The method is performed under an established set of reaction conditions using the control templates and a set of measurable amplicon parameters are obtained. The experimentally obtained parameters are plotted on an X-Y graph or other coordinate system and then a “curve” is generated, typically either manually or using one or more mathematical formula or algorithm, for example but not limited to graphing and/or line drawing software, linear regression analysis and similar mathematical calculations, computer algorithms, or the like. Once a standard curve have been generated for a given target region and primer set(s), experimental results obtained from a test (unknown) sample using the same primer set(s) under the same assay conditions can be evaluated using the standard curve and the degree to which the target region methylation in a sample can be extrapolated from the standard. The skilled artisan will appreciate that a “curve” can actually be a straight or substantially straight line or it can be curvilinear and assume a wide range of shapes.

Certain Exemplary Kits

The instant teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

In certain embodiments, kits comprise a target-specific primer pair, a first DNA polymerase, and a MSA. In certain embodiments, kits comprise a multiplicity of different target-specific primer pairs. In certain embodiments, kits further comprise a second DNA polymerase and an amplification primer. In some embodiments, kits comprise an amplification primer pair. In some embodiments, a forward amplification product primer, a reverse amplification product primer, or both primers of a primer pair comprise a universal priming sequence or the complement of a universal priming sequence. In some embodiments, kits comprise a forward primer, a reverse primer, or a forward primer and a reverse primer that further comprises a reporter group. In some such embodiments, the reporter group of a forward primer of a primer pair is different from the reporter group of the reverse primer of the primer pair. In some embodiments, kits further comprise at least one of: a modifying agent, a reporter probe, an intercalating agent, a reporter group, and a control sequence, for example but not limited to an internal standard sequence such as a housekeeping gene or a polynucleotide ladder comprising molecular size or weight standards.

The current teachings, having been described above, may be better understood by reference to examples. The following examples are intended for illustration purposes only, and should not be construed as limiting the scope of the teachings herein in any way.

### EXAMPLE 1

### Preparation of Modified Sample

Samples comprising methylated human gDNA (Serologicals P/N S7821 “CpGenome™ Universal Methylated DNA, human male, Serologicals Corp.) or unmethylated human gDNA (Coriell #NA17143, Coriell Cell Repositories, Camden, N.J.) were obtained from commercial sources. An aliquot of each of the two samples was treated with a modifying agent in parallel. In one MicroAmp reaction tube (Applied Biosystems P/N N801-0580), 330 ng of the unmethylated gDNA (1 μL of a Coriell gDNA) was combined with 44 μL water (total, including water from the DNA sample) and 5 μL, of M-dilution buffer (Zymo P/N D5001-2, Zymo Research, Orange, Calif.). The second MicroAmp reaction tube contained 330 ng of methylated gDNA (3.3 μL of Serologicals methylated gDNA), 41.7 μL water (total, including water from the DNA sample), and 5 μL, of M-dilution buffer (Zymo P/N D5001-2). The tubes were incubated for 15 minutes at 37° C. While the gDNA samples were incubating, Zymo CT conversion reagent P/N D5001-1 was mixed with 210 μL of the M-dilution buffer and 750 μL of molecular biology grade, nuclease-free water (Sigma W4502) and the mixture was vortexed periodically over 10 minutes prior to use. An 100 μL aliquot of the freshly prepared modifying reagent was added to each of the MicroAmp tubes (total volume 150 μL) and the tubes were incubated in a thermocycler at 50° C. for approximately 12-16 hours.

The modified samples were diluted with 200-300 μL water and each solution was transferred to an assembled Microcon 100 device (Millipore P/N YM-100) and centrifuged at 2800 rpm for 18 minutes in an Eppendorf 5414 centrifuge. The filtrate was removed, 350 μL additional water was added to the upper chambers, and the assemblies were centrifuged at 2800 rpm for 15 minutes. This wash step was repeated one additional time, then 350 μL 0.1 M NaOH was added to the upper chambers and the assemblies were centrifuged at 2800 rpm for 15 minutes. The filtrate was removed, 350 μL water was added to the upper chambers and the assemblies were centrifuged at 2800 rpm for 15 minutes. Fifty μL of TE buffer was added to the upper chambers and mixed by pipetting up and down several times. The assemblies were allowed to stand for about 5 minutes at room temperature, then the assemblies were inverted to collect the modified sample in TE buffer in Eppendorf tubes.

### EXAMPLE 2

### Evaluating the Degree of Methylation of a p15 Target Region

To evaluate the degree of methylation of a target region in the promoter region of the p15 tumor-suppressor gene (INK4B), a section of the published sequence (GenBank #S75756) was analyzed using “MethPrimer” software (found on the internet at: urogene.org/methprimer) to identify possible forward and reverse target-specific primer sequences. Because some DNA polymerases amplify homopolymer stretches inefficiently, the target region preferably does not contain homopolymer stretches, for example but not limited to, greater than 9T's or A's. A 289 base pair target region without a homopolymer stretch was identified. The bisulfite converted fully methylated target region comprised the sequence: TAGGTTTTTTAGGAAGGAGAGAGTGCGTCGGAGTAGCGTGGGAAAGAAGGGAAGAGTGTCGTTA AGTTTACGGTTAACGGTGGATTATTCGGGTCGTTGCGCGTTTGGGGGTTGCGGAATGCGCGAG GAGAATAAGGGTATGTTTAGTGGGGGCGGTAGCGATGAGGGTTTGGTTAGCGTCGCGGCGCG GGGATTAGTGGAGAAGGTGCGATAGTTTTTGGAAGTCGGCGCGGATTTTAACGGAGTTAATCG TTTCGGGAGGCGCGCGATTTAGGTAGTTGGGGTTTTAG (SEQ ID NO:1). The thirty CG sites are shown underlined and the upstream and downstream target flanking regions shown in italics. Due to bisulfite conversion, all of the underlined CG sites should have been converted to TG in the target region of the modified sample obtained from the unmethylated gDNA starting material. The forward primer of the exemplary p15 target-specific primer pair comprised the sequence: (FAM)TAGGTTTTTTAGGAAGGAGAGAGTG (SEQ ID NO:2) and included the fluorescent reporter group 6-FAM on its 5′-end (shown as “FAM”); the first target-specific portion is shown underlined. The reverse target-specific primer of the exemplary primer pair comprised the sequence: CAGGAAACAGCTATGACCCCTAAAACCCCAACTACCTAAATC (SEQ ID NO:3); the second target-specific portion is shown underlined and the upstream primer-binding site, in this illustration an M13 universal priming sequence, is shown in italics.

A portion of each of the two modified samples from Example 1 were combined with the target-specific primer pair in two separate first amplification compositions. Each of the first amplification compositions contained 1 μL AmpliTaq Gold 10× buffer (Applied Biosystems), 0.8 μL of a nucleotide mix (2.5 mM dATP, dCTP, dGTP, and dTTP), 0.8 μL MgCl₂) (25 mM), 0.2 μL AmpliTaq Gold DNA polymerase (Applied Biosystems), 0.25 μL forward target-specific primer (5 μM), 0.25 μL, reverse target-specific primer (5 μM), 0.5 μL, of one of the modified sample (10 ng/μL), and 6.2 μL water (Sigma). To generate first amplicons, the first amplification reactions were subjected to a thermocycling profile of 95° C. for 11 minutes, 40 cycles of (97° C. for 5 seconds, 57° C. for two minutes, and 72° C. for 45 seconds), then cooled to 4° C.

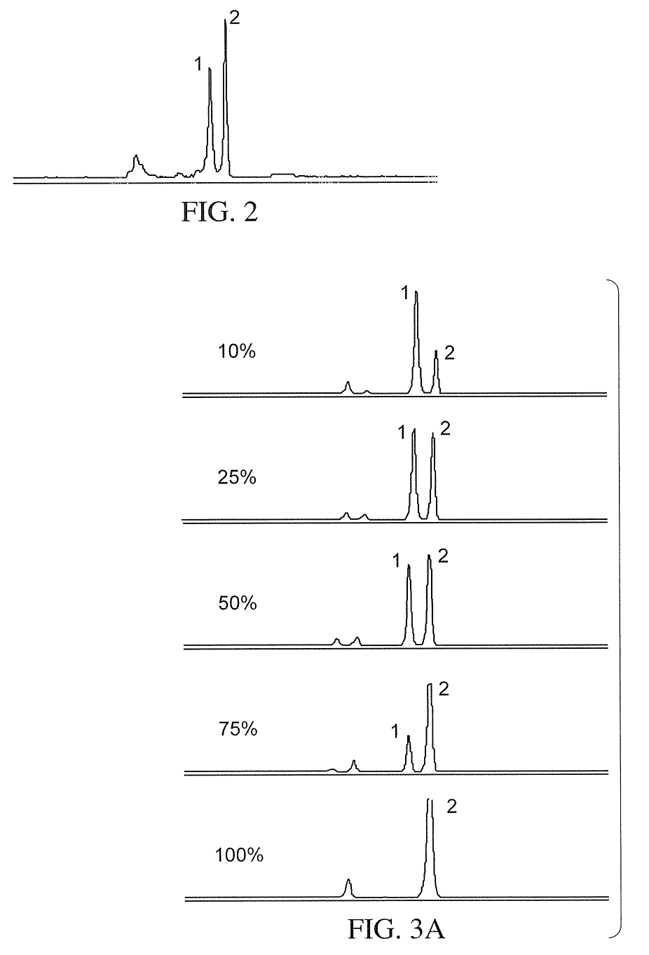

A 1.2 μL aliquot of each of the first amplification compositions comprising the first amplicon were mixed with 12 μL HiDi™ Formamide (P/N 4311320 ) containing 5-10% GeneScan™-500 ROX™ size standard (P/N 401734). To denature the DNA, the formamide mixture was heated at 95° C. for 5 minutes prior to analysis by capillary electrophoresis. Each of the amplicons was analyzed on an ABI PRISM® 310 Genetic Analyzer in 36 cm×50 micron capillaries comprising POP-4 polymer and the run module was GS POP4 (1 mL) A. After obtaining fragment analysis data on the individual amplicons derived from the methylated and unmethylated samples, equal volumes of the two first amplicon-formamide solutions were combined and co-injected in an ABI PRISM® 310 Genetic Analyzer (Applied Biosystems) to analyze the difference in migration rate of the two amplicons. As shown in FIG. 2, the amplicon peak derived from the methylated gDNA (“1”) migrated more slowly than the amplicon peak derived from the unmethylated gDNA (“2”). By comparing the migration rates of the two amplicon peaks with the GeneScan™-500 ROX™ control sequence (not shown), it was determined that the amplicon peak derived from the methylated gDNA migrated closer to the expected size. Thus, at least under these electrophoresis conditions, the methylated and unmethylated p15 target regions can be distinguished and their corresponding degree of methylation (in this illustrative embodiment, methylated vs. unmethylated) can be determined. Those in the art will appreciate that by modifying certain run conditions, for example but not limited to, electrophoresis temperature, polymer formulation, buffer type, buffer concentration, buffer pH, capillary length, the presence or absence of denaturing agents, voltage, and so forth, it may be possible to further increase or decrease the resolution of the methylated and unmethylated amplicon peaks.

### EXAMPLE 3

### Evaluation of Combined Methylated and Unmethylated Samples

Modified methylated and unmethylated gDNA samples were obtained, essentially as described in Example 1, except that a different unmethylated gDNA sample, Coriell #NA17136 (Coriell Cell Repositories), was used. The same p15 target region was amplified and analyzed, this time using a different p15 target-specific primer pair that included a forward target-specific primer with the sequence: (6-FAM)TGTAAAACGACGGCCAGTTAGGTTTTTTAGGAAGGAGAG (SEQ ID NO:4), including a first target-specific portion (underlined) and an upstream tail portion, comprising a FAM fluorescent reporter group at the 5′-end of the primer and a first primer binding site, an M13 universal forward priming site in this example; and a reverse target-specific primer with the sequence: GTTTCTTCAGGAAACAGCTATGACCCTAAAACCCCAACTACCTAAA (SEQ ID NO:5), including a second target-specific portion (underlined) and an upstream tail portion comprising a second primer-binding site, an M13 universal reverse priming sequence in this example, and an exemplary “PIGtail” sequence (shown in italics) at its 5′-end (Brownstein et al, BioTechniques, 20(6): 1004-10, 1996).

Mixtures of the modified methylated sample and the modified unmethylated sample were prepared as shown in Table 1. The modified methylated and modified unmethylated samples were not quantitated, so the percentages shown were estimated ratios, based on the assumptions that equal percent recoveries of the modified methylated sample and modified unmethylated sample were obtained after bisulfite-conversion and that the final DNA concentration of each was approximately 10 ng/μL.

TABLE 1

| ratio of methylated:unmethylated in | combined volumes |
|---|---|
| percent | (total volume: 7.5 μL) |
| 100% modified methylated sample(“Me”) | 7.5 μL Me |
| 100% modified unmethylated | 7.5 μL UnMe |
| sample (“UnMe”) | |
| 10% UnMe:90% Me | 0.75 μL UnMe + 6.75 μL Me |
| 25% UnMe:75% Me | 1.87 μL UnMe + 5.62 μL Me |
| 50% UnMe:50% Me | 3.75 μL UnMe + 3.75 μL Me |
| 75% UnMe + 25% Me | 5.62 μL UnMe + 1.87 Me |
| 90% UnMe 10% Me | 6.75 μL UnMe + 0.75 μL Me |

A series of first amplification compositions was formed in 7 MicroAmp tubes, each comprising 1 μL AmpliTaq Gold 10× buffer, 0.8 μL, of a dNTP mix (comprising 2.5 mM each of dATP, dCTP, dGTP, and dTTP), 0.8 μL MgCl₂ (25 mM), 0.2 μL AmpliTaq Gold DNA polymerase (5 μL; Applied Biosystems), 0.25 μL forward target-specific primer (5 μM), 0.25 μL reverse target-specific primer (5 μM), 0.5 μL of one of the modified samples mixes shown in Table 1 (˜10 ng/μL), and 6.2 μL water (Sigma). First amplicons were generated by subjecting the first amplification compositions to 95° C. for 5 minutes, 31 amplification cycles of (95° C. for 30 seconds, 60° C. for 2 minutes, and 72° C. for 45 seconds, 4 cycles of (95° C. for 30 seconds, 60° C. for 2 minutes, and 72° C. for 10 minutes), and then cooling to 4° c. The second set of cycling conditions was designed to enhance non-templated addition of an A nucleotide, sometimes referred to as the “Clark reaction” (see, e.g., Clark, Nucl. Acids Res. 16(20):9677, 1988; and Brownstein et al., BioTechniques 20(6):1004-10, 1996).

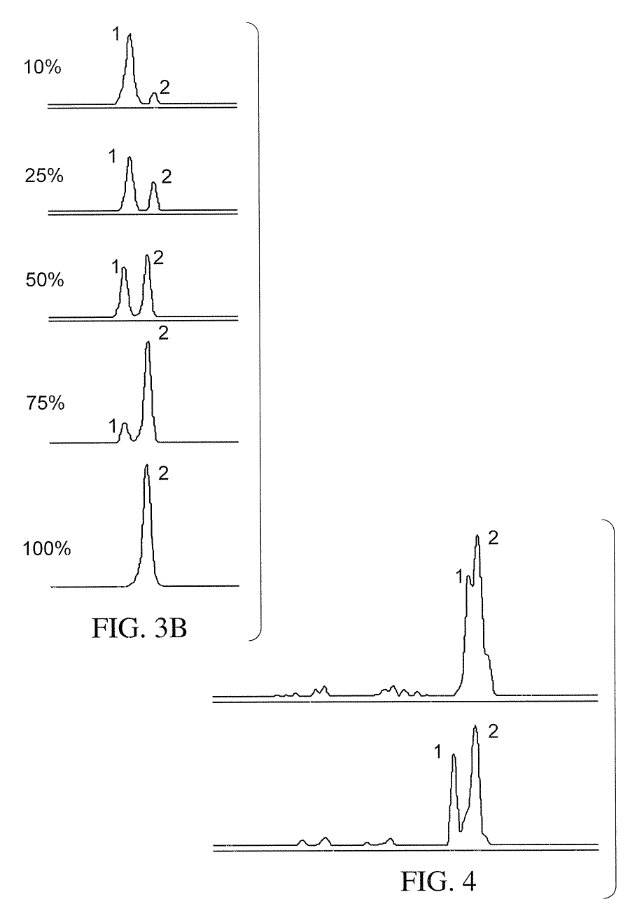

To each well of a clean MicroAmp Optical 96-well reaction plate (Applied Biosystems P/N N801-0560), 1.2 μL of one of the first amplification compositions comprising first amplicons was mixed with 12 μL HiDi™ Formamide (Applied Biosystems P/N 4311320 ) containing 5-10% GeneScan™-500 ROX™ size standard (Applied Biosystems P/N 401734). The reaction plate was covered with a 96 well 3100 Genetic Analyzer plate septa (Applied Biosystems P/N 4315933). The plate containing the first amplicon-formamide mixtures was heated at 95° C. for 5 minutes prior to analysis, to denature the DNA. Each of these mixtures was analyzed using an ABI PRISM® 3100 Genetic Analyzer using POP-4 polymer, a run module setting of GeneScan36_POP4DefaultModule, and an analysis module setting of GS500FragAnal.gsp, using a 16 capillary array, comprising 36 cm×50 micron capillaries. The results are shown in FIG. 3A and Table 2.

TABLE 2
Evaluation of the p15 amplicon from mixed modified samples

| p15 Amplicon | area of UnMe amplicon peak relative to the area |
|---|---|
| Mix | under both the Me and UnMe amplicon peaks in % |
| 10% UnMe | 25% |
| 25% UnMe | 47% |
| 50% UnMe | 56% |
| 75% UnMe | 77% |

To evaluate the degree of methylation of a target region in the p16 tumor-suppressor gene (INK4A), a section of the published p16 sequence (GenBank #X94154) was analyzed using MethPrimer. A 261 base pair target region, comprising 28 CG pairs, was identified. A p16 target-specific primer pair was evaluated comprising a forward primer with the sequence: (6-FAM)-TGTAAAACGACGGCCAGTGGTTGGTTGGTTATTAGAG (SEQ ID NO:6), including a first target-specific portion (underlined) and an upstream tail portion, comprising a FAM fluorescent reporter group at the 5′-end of the primer (shown as “6-FAM”) and a first primer-binding site, an M13 universal forward priming site in this example; and a reverse target-specific primer with the sequence: GTTTCTTCAGGAAACAGCTATGACCCCCTCTACCCACCTAAAT (SEQ ID NO:7), including a second target-specific portion (underlined) and a second tail portion comprising a second primer-binding site, an M13 universal reverse priming sequence in this example, and an exemplary “PIGtail” sequence (shown in italics) at its 5′-end. The p16 first amplicons were generated and analyzed using the method described in this example. The results are shown in FIG. 3B and Table 3.

TABLE 3
Evaluation of the p16 first amplicons from mixed modified samples

| p16 Amplicon | area of UnMe amplicon peak relative to the area |
|---|---|
| Mix | under both the Me and UnMe amplicon peaks in % |
| 10% UnMe | 8.7% |
| 25% UnMe | 29% |
| 50% UnMe | 53% |
| 75% UnMe | 85% |

Those in the art will appreciate that such mixed sample amplicon data can be used to generate standard curves, among other things, and the degree of methylation of a corresponding target region in a test (“unknown”) sample comprising, for example, a mixed cell population can be determined by extrapolation from the curve.

### EXAMPLE 4

### Evaluation of the SRBC Amplicon Using MSA Incorporation

To evaluate the use of MSA incorporation in determining the degree of target region methylation, a target region in the human SRBC gene (serum deprivation response factor (sdr)-related gene product that binds to c-kinase, “SRBC”) was analyzed. SRBC reported binds with the product of the breast cancer susceptibility gene BRCA1 (Xu et al., Cancer Res. 61:7943-49, 2001). The sequence of the SRBC target region is: TGGGGTTAATAGGTTTTTTAGTAGGTTTTCGGCGCGGGATAGCGGAAGGCGAAACGTTTTTAAGA GATTTCGTTGTTAATATTTTTACGTTTTCGCGTTTTTTCGTCGTTTTAGAAGGTTAATTTCGTTTG TTTGAGTTATAGTTGGAGTT (SEQ ID NO:8), wherein the CpG sites are shown underlined and the target flanking sequences are shown in italics. The SRBC-specific primer pair used in this example included a forward SRBC-specific primer comprising the sequence: TGTAAAACGACGGCCAGTTGGGGTTAATAGGTTTTTTAGTAGG (SEQ ID NO:9), including a first target-specific portion (underlined) and a first tail portion comprising a first primer-binding site; and a reverse SRBC-specific primer comprising the sequence: GTTTCTTCAGGAAACAGCTATGACCAACTCCAACTATAACTCAAACAAAC (SEQ ID NO:10) including a second target-specific portion (underlined) and a second tail portion comprising a second primer-binding site and an exemplary PIGtail sequence (shown in italics), located at the 5′-end of the reverse primer.

Two different first amplification compositions, each comprising 2.5 μL AmpliTaq Gold 10× buffer, 2 μL, of an appropriate NTP mix (comprising 2.5 mM each of dATP, dCTP, dGTP, and dTTP), 2 μL MgCl₂ (25 mM), 0.5 μL AmpliTaq Gold DNA polymerase, 0.625 μL SRBC-specific forward primer (5 μM), 0.625 μL SRBC-specific reverse primer (5 μM), 15.5 μL, water, and either (a) 1.25 μL modified mixed sample (comprising a 50:50 mixture of modified methylated and modified unmethylated samples), or (b) 1.25 μL, modified unmethylated sample, were formed in two MicroAmp reaction tubes (total volumes were 25 μL). The first amplification compositions were heated to 95° C. for five minutes, then subjected to 31 cycles of amplification, each comprising (97° C. for 5 seconds, 60° C. for two minutes, and 72° C. for 72 seconds), 4 cycles of amplification, each comprising (97° C. for 5 seconds, 60° C. for two minutes, and 72° C. for ten minutes), then the first amplification compositions were cooled to 4° C. The first amplicons were purified using a QIAQuick PCR Clean-Up Kit (P/N 28104, Qiagen Sciences, MD) according to the manufacturer's protocol and the purified amplicons recovered in a final volume of 30 μL EB buffer (10 mM Tris-HCl, pH 8.5). An aliquot of each of the purified first amplicons in EB buffer was further purified (doubly-purified) by mixing 10 μL of one first amplicons in EB with 1 μL shrimp alkaline phosphatase (USB Corp., P/N 70092X, 1 unit/μL) and then incubating at 37° C. for one hour and then at 72° C. for 15 minutes, in parallel.

Four different second amplification compositions were prepared. Three of the different second amplification compositions comprised 0.5 μL 10× ThermoPol buffer (New England Biolabs, Beverly, Md.), 0.5 μL DMSO, 0.05 μL Vent® DNA polymerase (New England Biolabs), 0.25 μL amplification product-specific forward primer (5 μM, comprising the sequence: (6-FAM)-TGTAAAACGACGGCCAGT (SEQ ID NO:11), including the fluorescent reporter group FAM at its 5′-end, 1 μL, of the doubly-purified mixed first amplicons, and either (a) 0.7 μL water, 1 μL, α-thio-dCTP (1 mM, P/N N-8002 TriLink Biotechnologies, San Diego, Calif.; an illustrative mobility shifting analog of dCTP) and 1 μL of an dNTP mix comprising dATP, dGTP, and dTTP (1 mM each), (b) 0.7 μL water, 1 μL biotin-aha-dCTP (1 mM, P/N B32772, Molecular Probes; an illustrative mobility shifting analog of dCTP) and 1 μL of an dNTP mix comprising dATP, dGTP, and dTTP (1 mM each), or (c) 1.7 μL, water and 1 μL, of an dNTP mix comprising dATP, dCTP, dGTP, and dTTP (1 mM each). The fourth second amplification composition comprised 0.5 μL, 10× ThermoPol buffer, 0.5 μL DMSO, 0.05 μL Vent® DNA polymerase, 0.25 μL amplification product-specific forward primer (5 μM, comprising the sequence: (6-FAM)-TGTAAAACGACGGCCAGT (SEQ ID NO:12), including the fluorescent reporter group FAM at its 5′-end), 1 μL of the doubly-purified unmethylated first amplicons, 1 μL, biotin-aha-dCTP (1 mM) and 1 μL of a dNTP mix comprising dATP, dGTP, and dTTP (1 mM each). The final volume of each second amplification composition was 5.0 μL. FAM-labeled second amplicons were generated by subjecting the second amplification compositions in parallel to 96° C. for 1 minute, and 5 amplification cycles comprising (96° C. for 10 seconds, 50° C. for 5 seconds, and 60° C. for 4 minutes), then the second amplification

compositions comprising second amplicons was cooled to 4° C.

1.2 μL, of each of the four thermocycled second amplification compositions was added to a separate well of a MicroAmp Optical 96-well reaction plate (Applied Biosystems P/N N801-0560), one well for each of the second amplicons, and each was mixed with 12 μL HiDi™ Formamide containing 5-10% GeneScan™-500 ROX™ size standard. The reaction plate was covered with a 96 well 3100 Genetic Analyzer plate septa. To denature the DNA, the second amplicon-formamide mixtures, the plate was heated at 95° C. for 5 minutes prior to analysis. Each of the mixtures was analyzed on an ABI PRISM® 3100 Genetic Analyzer using a 16 capillary array, comprising 36 cm×50 micron capillaries and POP-4 polymer. The run module was GeneScan36_POP4DefaultModule and analysis module was GS500FragAnal.gsp.

The top panel of FIG. 4 shows the results obtained using the conventional NTP mix, with the methylated amplicon peak (1), substantially overlapping the unmethylated amplicon peak (2). The bottom panel of FIG. 4 shows the results obtained using the NTP mix comprising dATP, dGTP, dTTP, and alpha-thio-dCTP. The distance between the methylated amplicon peak (1) and the unmethylated amplicon peak (2) is increased, due to the presence of mobility shifting dCTP analog in the methylated amplicon but not the unmethylated amplicon (excluding any analog in the tail portion). The alpha-thio-dCTP is incorporated in the methylated amplicon wherever the target region comprises a 5 mC, resulting in a shift in the mobility of the methylated amplicon.

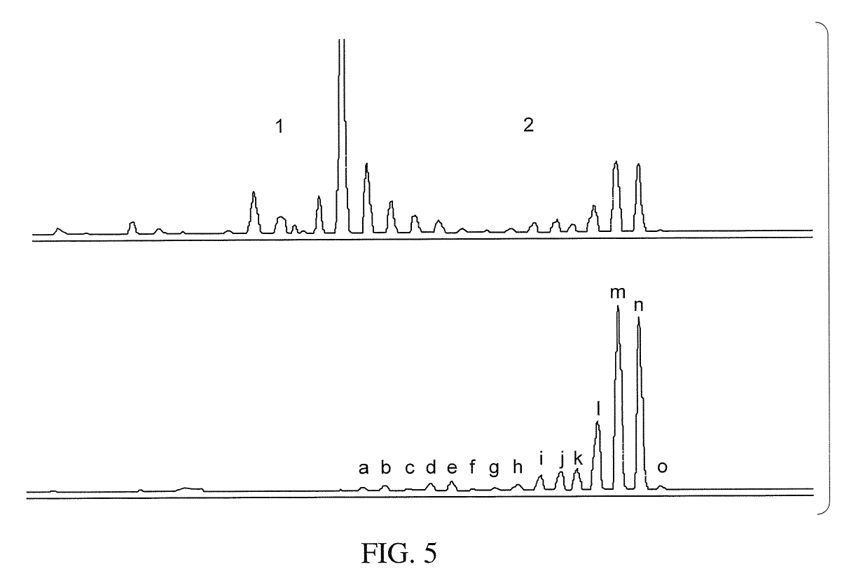

The top panel of FIG. 5 shows the results obtained using the mixed modified sample (50% modified methylated sample and 50% modified unmethylated sample) and the dNTP mix comprising biotin-aha-dCTP. As shown in the upper panel, a series of amplicon peaks were observed, with the unmethylated amplicon peaks (1), migrating more slowly than the methylated amplicon peaks (2). The bottom panel of FIG. 5 shows the results obtained using the modified methylated sample and the dNTP mix comprising dATP, dGTP, dTTP, and biotin-aha-dCTP. Thus, the amplicon peaks seen in the bottom panel correspond to second amplicons derived only from the fully methylated SRBC target region. The methylated amplicon peaks comprised forward second amplicons that should contain 17 biotin-aha-dCTPs, due to the 13 CG pairs in the target region and the 4 Cs in the primer. While not intending to be limited by a particular theory, it may be that the multiple peaks seen for the mixed methylated amplicon and unmethylated amplicon (upper panel) and the methylated amplicon alone (lower panel) are due to the presence of dCTP as a contaminant in the biotin-aha-dCTP preparation, which competes with the MSA during the second amplification reaction. As seen in the lower panel of FIG. 5, however, a family of related amplicon peaks (fifteen relatively evenly spaced peaks, designated by the letters “a” through “o”) derived from the methylated SRBC target region was detected using these exemplary reaction conditions and analyzing technique.

The compositions, methods, and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

Although the disclosed teachings has been described with reference to various applications, methods, and kits, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

We claim:

1. A method for determining the degree of methylation of at least one genomic DNA (gDNA) target region in a sample comprising,
exposing the sample to a modifying agent to obtain a modified sample;
forming a first amplification composition comprising at least some of the modified sample, a target-specific primer pair for each target region, and a first DNA polymerase;
subjecting the first amplification composition to at least one first cycle of amplification to generate at least one first amplification product;
analyzing at least part of the at least one first amplification product; and
determining the degree of methylation of at least one target region.

2. The method of claim 1, wherein the analyzing comprises electrophoresis.

3. The method of claim 1, wherein the first amplification composition further comprises a reporter probe, an intercalating agent, or a reporter probe and an intercalating agent.

4. The method of claim 1, wherein the modifying agent modifies at least one unmethylated target nucleotide to a modified nucleotide, but does not modify at least one methylated target nucleotide to a modified nucleotide.

5. The method of claim 1, wherein the at least one gDNA target region comprises a multiplicity of different gDNA target regions and the at least one target-specific primer pair comprises a multiplicity of different target-specific primer pairs.

6. The method of claim 5, wherein the first amplification composition comprises a multiplicity of different first amplification compositions, each comprising (a) at least some of the modified sample and (b) one target-specific primer pair, two different target-specific primer pairs, three different target-specific primer pairs, four different target-specific primer pairs, five different target-specific primer pairs, or six different target-specific primer pairs.

7. The method of claim 1, wherein at least one primer pair comprises a forward primer comprising a first reporter group, a reverse primer comprising a second reporter group, or a forward primer comprising a first reporter group and a reverse primer comprising a second reporter group, wherein the first reporter group and the second reporter group are the same or different.

8. The method of claim 1, wherein the at least one first cycle of amplification comprises a multiplicity of cycles of amplification.

9. A method for determining the degree of methylation of at least one gDNA target region in a sample comprising:
exposing the sample to a modifying agent to obtain a modified sample;
forming a first amplification composition comprising at least some of the modified sample, a target-specific primer pair for each target region, a mobility shifting analog, and a first DNA polymerase;
subjecting the first amplification composition to at least one first cycle of amplification to generate at least one first amplification product comprising at least one mobility shifting analog;
analyzing at least part of the first amplification product; and
determining the degree of methylation of at least one target region.

10. The method of claim 9, wherein the determining comprises identifying the number of methylated target nucleotides in at least one target region.

11. The method of claim 9, wherein the at least one mobility shifting analog comprises at least one of: a biotin moiety, a fluorophore, a hydrocarbon, a heterocyclic derivative of a hydrocarbon, a boranotriphosphate, a polyethylene glycol moiety, and a thiotriphosphate.

12. The method of claim 9, wherein the analyzing comprises electrophoresis.

13. The method of claim 9, wherein the first amplification composition further comprises a reporter probe, an intercalating agent, or a reporter probe and an intercalating agent.

14. The method of claim 9, wherein the modifying agent modifies at least one unmethylated target nucleotide to a modified nucleotide, but does not modify at least one methylated target nucleotide to a modified nucleotide.

15. The method of claim 9, wherein the at least one gDNA target region comprises a multiplicity of different gDNA target regions and the at least one target-specific primer pair comprises a multiplicity of different target-specific primer pairs.

16. The method of claim 15, wherein the first amplification composition comprises a multiplicity of different first amplification compositions, each comprising (a) at least some of the modified sample and (b) one target-specific primer pair, two different target-specific primer pairs, three different target-specific primer pairs, four different target-specific primer pairs, five different target-specific primer pairs, or six different target-specific primer pairs.

17. The method of claim 9, wherein at least one primer pair comprises a forward primer comprising a first reporter group, a reverse primer comprising a second reporter group, or a forward primer comprising a first reporter group and a reverse primer comprising a second reporter group, wherein the first reporter group and the second reporter group are the same or different.

18. The method of claim 9, wherein the at least one first cycle of amplification comprises a multiplicity of cycles of amplification.

19. A method for determining the degree of methylation of at least one gDNA target region in a sample comprising:
exposing the sample to sodium bisulfite to obtain a modified sample;
forming a first amplification composition comprising at least some of the modified sample, a target-specific primer pair for each target region, at least one mobility shifting analog, and a first DNA polymerase, wherein at least one forward primer, at least one reverse primer or both primers of a target-specific primer pair comprises a fluorescent reporter group and wherein the at least one mobility shifting analog comprises at least one of: a biotin moiety, a fluorophore, a hydrocarbon, a heterocyclic derivative of a hydrocarbon, a boranotriphosphate, a polyethylene glycol moiety, and a thiotriphosphate;
subjecting the first amplification composition to at least one first cycle of amplification to generate at least one first amplification product comprising at least one mobility shifting analog;
analyzing at least part of the at least one first amplification product using capillary electrophoresis; and
determining the degree of methylation of at least one target region.

20. A method for determining the degree of methylation of at least one gDNA target region in a sample comprising:
exposing the sample to a modifying agent to obtain a modified sample;
forming a first amplification composition comprising at least some of the modified sample, a target-specific primer pair for each target region, and a first DNA polymerase;
subjecting the first amplification composition to at least one first cycle of amplification to generate at least one first amplification product;
forming a second amplification composition comprising at least some of the first amplification product, at least one first amplification product primer, a second DNA polymerase, and a mobility shifting analog;
subjecting the second amplification composition to at least one second cycle of amplification to generate at least one second amplification product comprising at least one mobility shifting analog;
analyzing at least part of the at least one second amplification product; and
determining the degree of methylation of at least one target region.

21. The method of claim 20, wherein the determining comprises identifying the number of methylated target nucleotides in at least one target region.

22. The method of claim 20, wherein the at least one mobility shifting analog comprises at least one of: a biotin moiety, a fluorophore, a hydrocarbon, a heterocyclic derivative of a hydrocarbon, a boranotriphosphate, a polyethylene glycol moiety, and a thiotriphosphate.

23. The method of claim 20, wherein the at least one first amplification product primer comprises a universal priming sequence or its complement.

24. The method of claim 20, wherein the at least one first amplification primer comprises at least one first amplification product primer pair.

25. The method of claim 24, wherein at least one of the primers of the at least one first amplification product primer pair comprises a universal priming sequence or its complement.

26. The method of claim 20, wherein the first DNA polymerase and the second DNA polymerase are the same or different.

27. The method of claim 20, wherein the analyzing comprises electrophoresis.

28. The method of claim 20, wherein the second amplification composition further comprises a reporter probe, an intercalating agent, or a reporter probe and an intercalating agent.

29. The method of claim 20, wherein the modifying agent modifies at least one unmethylated target nucleotide to a modified nucleotide, but does not modify at least one methylated target nucleotide to a modified nucleotide.

30. The method of claim 20, wherein the at least one gDNA target region comprises a multiplicity of different gDNA target regions and the at least one target-specific primer pair comprises a multiplicity of different target-specific primer pairs.

31. The method of claim 30, wherein the first amplification composition comprises a multiplicity of different first amplification compositions, each comprising (a) at least some of the modified sample and (b) one target-specific primer pair, two different target-specific primer pairs, three different target-specific primer pairs, four different target-specific primer pairs, five different target-specific primer pairs, or six different target-specific primer pairs.

32. The method of claim 20, wherein at least one first amplification product primer comprises a forward first amplification product primer comprising a first reporter group, a reverse first amplification product primer comprising a second reporter group, or a forward first amplification product primer comprising a first reporter group and a reverse first amplification product primer comprising a second reporter group, wherein the first reporter group and the second reporter group are the same or different.

33. The method of claim 20, wherein (a)(i) the subjecting the first amplification composition to at least one first cycle of amplification, (ii) the subjecting the second amplification composition to at least one second cycle of amplification, or (iii) the subjecting the first amplification composition to at least one first cycle of amplification and the subjecting the second amplification composition to at least one second cycle of amplification comprises (b) a multiplicity of cycles of amplification.

34. The method of claim 20, further comprising a Q-PCR reaction and wherein at least one amplification product further comprises a reporter probe-binding portion.

35. The method of claim 20, wherein the second cycle of amplification comprises Q-PCR, wherein at least one first amplification product comprises a reporter probe-binding portion, and wherein the second amplification composition further comprises at least one reporter probe.

36. A method for determining the degree of methylation of at least one gDNA target region in a sample comprising:
exposing the sample to sodium bisulfite to obtain a modified sample;
forming a first amplification composition comprising at least some of the modified sample, a target-specific primer pair for each target region, and a first DNA polymerase;
subjecting the first amplification composition to at least one first cycle of amplification to generate at least one first amplification product;
forming a second amplification composition comprising at least some of the first amplification product, at least one forward amplification product primer comprising a fluorescent reporter group, a permissive DNA polymerase, and a mobility shifting analog, wherein the at least one mobility shifting analog comprises at least one of: a biotin moiety, a fluorophore, a hydrocarbon, a heterocyclic derivative of a hydrocarbon, a boranotriphosphate, a polyethylene glycol moiety, and a thiotriphosphate;
subjecting the second amplification composition to at least one second cycle of amplification to generate at least one second amplification product comprising at least one mobility shifting analog;
analyzing at least part of the at least one second amplification product using capillary electrophoresis; and
determining the degree of methylation of at least one target region.

37. The method of claim 36, further comprising a Q-PCR reaction and wherein at least one amplification product further comprises a reporter probe-binding portion.

38. The method of claim 36, wherein the second cycle of amplification comprises Q-PCR, wherein at least one first amplification product comprises a reporter probe-binding portion, and wherein the second amplification composition further comprises at least one reporter probe.

39. A kit comprising a first DNA polymerase, a mobility shifting analog, and a target-specific primer pair for each target region.

40. The kit of claim 39, further comprising at least one of: a control sequence, a modifying agent, an amplification product primer, a permissive DNA polymerase, a reporter probe, an intercalating agent, and a reporter group.

41. The kit of claim 39, wherein the mobility shifting analog comprises at least one of: a biotin moiety, a fluorophore, a hydrocarbon, a heterocyclic derivative of a hydrocarbon, a boranotriphosphate, a polyethylene glycol moiety, and a thiotriphosphate.
